(19) United States
(12) Patent Application Publication
(10) Pub. No.: US 2011/0223146 A1
(43) Pub. Date: Sep. 15, 2011

(54) METHODS FOR PREPARATION AND USE OF MARROW INFILTRATNG LYMPHOCTYES (MILS)

(75) Inventors: Ivan Marques Borrello, Baltimore, MD (US); Kimberly Ann Noonan, Baltimore, MD (US)

(73) Assignee: THE JOHNS HOPKINS UNIVERSITY, Baltimore, MD (US)

(21) Appl. No.: 13/127,374

(22) PCT Filed: Nov. 3, 2009
(86) PCT No.: PCT/US2009/063077

§ 371 Date: May 3, 2011

Related U.S. Application Data
(60) Provisional application No. 61/110,768, filed on Nov. 3, 2008.

(51) Int. Cl. A61K 35/28; C12N 5/078; C12M 3/00; C12N 5/071; A61P 35/00

(52) U.S. Cl. 424/93.71; 435/325; 435/289.1

(57) ABSTRACT

The invention provides compositions comprising activated marrow infiltrating lymphocytes, methods of generating populations of marrow infiltrating lymphocytes, uses of the marrow infiltrating lymphocytes of the invention, and a culture device for use in cell culture, for example for use in generating populations of activated marrow infiltrating lymphocytes. In certain embodiments, the marrow infiltrating lymphocytes can be used as a cancer therapeutic.

### CROSS-REFERENCE TO RELATED APPLICATIONS

This application claims the benefit of U.S. Provisional Application Ser. No. 61/110,768, filed Nov. 3, 2008, the entire contents of which are incorporated herein by reference.

### STATEMENT OF RIGHTS TO INVENTIONS MADE UNDER FEDERALLY SPONSORED RESEARCH

The work was supported by in part, by NIH grants CA15396. The Government has some rights to the invention.

### BACKGROUND OF THE INVENTION

T cell immune responses can mediate a tumor-specific response in the appropriate setting. Unfortunately, the severe defects underlying immune responses in cancer-bearing hosts limit the efficacy of adoptive T cell transfer from such hosts. Ex-vivo activation of T cells with beads conjugated to CD3 and CD28 antibodies has been capable of activating and augmenting peripheral T cells in a non-specific polyclonal manner. However, a major concern is the absence of antigen-specificity of this approach.

In recent years, adoptive immunotherapy in cancer has shown measurable clinical activity yet many obstacles still exist to maximize its efficacy. For adoptive immunotherapy to be effective, T cells must overcome the intrinsic tolerogenic mechanisms that often limit immune responsiveness. Specifically they must be tumor-specific, capable of being expanded to clinically meaningful numbers, traffic to the tumor microenvironment upon infusion into the host, and kill the tumor upon its encounter. To date, multiple strategies have been attempted to increase the tumor specificity of adoptive immunotherapy). When antigen-specific lines or clones have been used, they require the need to identify and isolate peptides or tumor antigens thereby limiting this approach to a finite number of known tumor antigens and increasing the likelihood of recurrent disease associated with antigen-escape variants.

Much of the work pioneered by Rosenberg et al. at the National Cancer Institute has demonstrated the increased tumor specificity of tumor infiltrating lymphocytes (TILs) obtained from metastatic lesions in melanoma patients. Interestingly, despite reported clinical efficacy, TILs are only present in approximately half of all metastatic lesions and of those, they can only be expanded in a fraction of those patients. As such, although showing significant anti-tumor activity, the overall applicability of such an approach in a solid tumor such as metastatic melanoma is limited to a selected subset of patients in which TILs are actually present which normally represents less than 50% of all patients with metastatic disease.

Multiple myeloma is a plasma cell disorder that primarily involves the bone marrow of patients and still remains incurable with a median survival rate is 3 to 5 years post diagnosis despite numerous recent advances. The immunotherapeutic approaches utilized thus far include both vaccines such as: idiotype vaccines; and whole cell autologous vaccines coupled to a GM-CSF—secreting bystander cell line as well as adoptive immunotherapy T-cell based approaches utilizing activated and expanded peripheral blood lymphocytes. However, to date these approaches have failed to show significant clinical efficacy.

### SUMMARY OF THE INVENTION

The invention provides compositions comprising activated marrow infiltrating lymphocytes, methods of generating populations of marrow infiltrating lymphocytes, uses of the marrow infiltrating lymphocytes of the invention, and a culture device for use in cell culture, for example for use in generating populations of activated marrow infiltrating lymphocytes.

The invention provides methods for expanding marrow infiltrating lymphocytes (MILs) including obtaining bone marrow from a subject having a malignant cancer; contacting the bone marrow with a phosphodiesterase (PDE)-5 inhibitor; and contacting the bone marrow with anti-CD3 and anti-CD-28 antibodies, wherein the aMILs are expanded at least 30-, 40-, 50-, 60-, 70-, 80-, 90-, 100-fold, or more. In certain embodiments, the cancer is a hematological malignancy. In certain embodiments the bone marrow is contacted with a single dose of a PDE-5 inhibitor. In certain embodiments, the bone marrow is contacted with more than one dose, e.g., 2, 3, 4, 5, 6, etc. doses of PDE-5 inhibitor. In certain embodiments, the bone marrow is contacted with the anti-CD3 and anti-CD-28 antibodies for 5-14 days (e.g., 5, 6, 7, 8, 9, 10, 11, 12, 13, or 14 days). In certain embodiments, after contacting the bone marrow with the anti-CD3 and anti-CD28 antibodies, the antibodies are removed. In certain embodiments, the expansion of the MILs is continued after the removal of the antibodies. In certain embodiments, the antibodies are removed at the conclusion of MIL expansion. In certain embodiments, the antibodies are coated on beads to facilitate removal of the beads from the cells after expansion. For example, the beads can be metal and removed using a magnet. The beads are present at a ratio sufficient to insure activation of essentially all of the marrow infiltrating lymphocytes present in the bone marrow. The beads can be present in the culture at a ratio of 1:1 to 5:1; 2:1 to 4:1, or 2.5:1 to 3.5:1, or any values within the ranges, beads to cells.

The methods of expansion of the invention include the specific expansion of MILs, wherein the malignant cells are not expanded. For example, the number of malignant cells in the population is decreased by at least 50%, 60%, 70%, 80%, 90%, or 95%, or any value within the range of 50-99%, as compared to the number of malignant cells present in the bone marrow population prior to culturing as determined by flow cytometry staining with an anti-CD30 antibody. In certain embodiments, at the end of the MIL expansion, the number of malignant cells present is below the level of detection as determined by flow cytometry, essentially reducing the number of malignant cells by 100%.

In certain embodiments of the invention, the presence of the PDE-5 inhibitor increases the expansion of MILs relative to a control culture not containing a PDE-5 inhibitor.

In certain embodiments, the increase in expansion in the culture containing a PDE-5 inhibitor is at least 30%, 40%, 50%, 60%, 70%, 80%, 90%, or 100% (or any value from 30% to 100%) greater in a culture contacted with a PDE-5 inhibitor as compared to a culture not contacted with a PDE-5 inhibitor.

In certain embodiments, the MILs are expanded, at least part of the time, in a culture device of the invention. A culture device of the invention includes a rigid, stable, round bottom, closed container of sufficient volume for expanding bone marrow in a volume of at least 20 ml of cells and growth media.

In certain embodiments of the invention, the bone marrow is treated to substantially remove the neutrophils and red blood cells from the bone marrow prior to contacting the bone marrow with the anti-CD3 and anti-CD-28 antibodies. In certain embodiments, the neutrophils and red blood cells are substantially removed by density centrifugation. In certain embodiments, independently the number of each neutrophils and red blood cells is reduced by at least at least 75%, at least 80%, at least 85%, at least 90%, at least 95%, at least 98%, or all detectable red blood cells and neutrophils as compared to the bone marrow sample obtained from the subject. It is understood that the red blood cells and neutrophils need not be removed to the same degree (i.e., the residual amount of red blood cells and neutrophils present in the bone marrow need not be the same).

In certain embodiments, the bone marrow is not enriched for T-cells after substantially removing the neutrophils and red blood cells. That is, the bone marrow is not selected using cell surface markers to select specific cell types for expansion in culture. Particularly, the T-cells are not isolated from the bone marrow prior to expansion in culture.

In certain embodiments, the bone marrow cells are expanded in a static culture for at least 2, 3, 4, 5, 6, 7, 8, 9, or 10 days.

In certain embodiments of the invention, the PDE-5 inhibitor is present at a concentration that is effective blocking MDSC function by at least 50%, 60%, 70%, 80%, 90%, or more. In the methods provided herein, MDSC function is understood as the inhibition of expansion of activated MILs.

The expanded MILs of the invention can be used for any purpose. In an embodiment, the aMILs of the invention, or MILs prepared by any other method, can be used for the treatment of cancer by administering aMILs of to a subject, preferably the subject from which the bone marrow was obtained. In certain embodiments, the cancer is a hematological malignancy. In certain embodiments the MILs are administered once. In certain embodiments, the MILs are administered more than once, e.g., 2, 3, 4, 5, 6, 7, etc. times.

In certain embodiments, the MILs are administered in conjunction with other cancer therapies. For example, the MILs can be administered in conjunction with a bone marrow transplant. The MILs can be administered in conjunction with lymphoablation which can be performed for the treatment of cancer and/or to provide space for engraftment of the MILs in the subject.

The invention provides for the preparation of a medicament for the treatment of cancer, for example a hematological malignancy.

The invention provides compositions including MILs prepared by any of the methods provided herein in a pharmaceutically acceptable carrier.

The invention provides devices for culturing cells for administration to a human subject including an enclosed cell culture container comprising a smooth, rigid, rounded bottom surface; a first port and a second port operably linked to the cell culture container; and a support to maintain the cell culture container in a fixed position to allow for static cell culture. In certain embodiments, the volume of the culture is at least 40 ml, 50 ml, 60 ml, 70 ml, 80 ml, 90 ml, 100 ml, 110 ml, 120 ml, 130 ml, 140 ml, 150 ml, 160 ml, 170 ml, 180 ml, or 190 ml. In certain embodiments, the volume of culture is no more than 200 ml, 225 ml, 250 ml, 275 ml, 300 ml, 350 ml, 400 ml, 450 ml, 500 ml, 1000 ml, 1250 ml, 1500 ml, or 2000 ml. In certain embodiments, the volume of the culture is any of the minimum values provided paired with any of the maximum values. In certain embodiments, the volume of the culture is any value from 40 ml to 2000 ml.

In certain embodiments, at least one port includes a connector. In certain embodiments, at least one port a valve. In certain embodiments, the cell container includes more than two ports, e.g., 3, 4, 5, 6, 7, 8, 9, 10 or more ports.

In certain embodiments, the cell container is made at least partially of a flexible material. In certain embodiments, the cell container is made at least partially of a rigid material. In certain embodiments, the cell container is gas permeable. In certain embodiments the cell container can be sterilized. In certain embodiments, the cell container meets the requirements of the FDA and other regulatory bodies for use in culturing cells for administration to a human or animal.

The invention provides methods for use of the culture device of the invention. Methods include introducing cells into the cell container; introducing media into the cell container, introducing air into the cell container as needed to make the cell container rigid; placing the cell container on the support; and culturing the cells in a static culture. In certain embodiments, when material is introduced into the container through a port, air is expelled through another port. In certain embodiments, the cells for culture in the container include bone marrow or fractionated bone marrow.

The invention provides kits including one or more components of cell culture device of the invention.

### BRIEF DESCRIPTION OF THE DRAWINGS

FIG. 1 shows the effect of adoptive immunotherapy with aMILs on tumor-free survival. Mice were challenged with H929 and then given T cells as indicated and followed for survival. No difference was observed with the three different doses of activated MILs used, so the data are shown in aggregate.

FIG. 2 shows the distribution of T cells and tumor in bone marrow. Bone marrows of mice that received either aPBLs or aMILs were analyzed for human CD3 T cells as well as human CD138 plasma cells.

FIG. 3 shows that Activated MILs eradicate pre-established disease.

FIGS. 4A-C show the effect of SLx-2101 on aMILs Expansion. (A) MILs from myeloma patients were expanded with anti-CD3/CD28 beads in the presence or absence of the PDE-5-inhibitor SLX-2101 for 7 days. Fold expansion was determined by flow cytometery; (B) Fold expansion and (C) tumor specificity of aMILs was compared in without PDE-5 inhibitor, with PDE-5 inhibitors Viagra® or SLx-2102 from Surface Logix.

FIGS. 5A and B show a front view (A) and a cross-sectional view at a vertical plane at (B) of a culture device of the invention.

### DEFINITIONS

An “agent” is understood herein to include a therapeutically active compound or a potentially therapeutic active compound, e.g., an antioxidant. An agent can be a previously known or unknown compound. As used herein, an agent is typically a non-cell based compound, however, an agent can include a biological therapeutic agent, e.g., peptide or nucleic acid therapeutic, e.g., cytokine, antibody, etc.

The term “amelioration” refers to a reduction of at least one sign and/or symptom of a specific disease or condition. Treatment refers to reduction of at least one sign and/or symptom of a disease or condition to reduce or eliminate at least one sign and/or symptom of the disease or condition, or to prevent progression of the disease or condition. Amelioration and treatment need not be considered separate interventions, but instead can be considered a continuum of therapeutic interventions.

As used herein, “cancer” is understood as a group of diseases or conditions characterized by malignant hyperplasia and/or neoplasia. Types of cancer include, but are not limited to: carcinoma, which includes malignant tumors derived from epithelial cells, for example cancers of the breast, prostate, lung and colon; sarcoma which includes malignant tumors derived from connective tissue, or mesenchymal cells; lymphoma and leukemia, which include malignancies derived from hematopoietic (blood-forming) cells; germ cell tumors, which includes tumors derived from totipotent cells, most often found in the testicle and ovary in adults; and in fetuses, babies, and young children most often found on the body midline, particularly at the tip of the tailbone; and blastic tumor or blastoma which includes tumors which resembles an immature or embryonic tissue. Many of these tumors are most common in children.

As used herein, “changed as compared to a control reference sample” is understood as having a level or activity of an analyte, or in a whole organism change of physical characteristics or signs or symptoms of a disease, to be detected at a level that is statistically different than a sample from a normal, untreated, or control sample. Methods to select and test control samples are within the ability of those in the art. Control samples typically include a cell or an animal of the same type that has not been contacted with an active agent or been subjected to a particular treatment, and has optionally been contacted with a carrier or subjected to a sham treatment. Control samples also include a cell or an animal not subjected to an agent or treatment to induce a specific disease or condition.

As used herein, “compound” or “pharmaceutical compound” of the invention and the like include activated MILs prepared by the methods of the invention. The cells can be administered alone or in conjunction with other pharmaceutical agents and compositions, either pharmaceutically active or carrier agents and compositions.

“Contacting a cell” is understood herein as providing an agent to a cell, in culture or in an animal, such that the agent can interact with the surface of the cell, potentially be taken up by the cell, and have an effect on the cell. The agent can be delivered to the cell directly (e.g., by addition of the agent to culture medium or by injection into the cell or tissue of interest), or by delivery to the organism by an enteral or parenteral route of administration for delivery to the cell by circulation, lymphatic, or other means.

As used herein, “detecting”, “detection” and the like are understood that an assay performed for identification of a specific analyte in a sample or a change in a subject of at least one sign or symptom of a disease, expression of a protein or gene, including a reporter construct. The amount of analyte detected in the sample or change in a subject can be none or below the level of detection of the assay or method.

The term “detectable label” is understood as a chemical modification, binding agent, or other tag that can be readily observed, preferably in a quantitative manner, such as a fluorescent tag that has specific wavelengths of absorption and emission to allow detection of the compound associated with the detectable label.

The terms “disease” or “condition” are commonly recognized in the art and designate the presence of at least one sign and/or symptom in a subject or patient that are generally recognized as abnormal. Diseases or conditions may be diagnosed and categorized based on pathological changes. Signs may include any objective evidence of a disease such as changes that are evident by physical examination of a patient or the results of diagnostic tests that may include, among others, laboratory tests. Symptoms are subjective evidence of disease or a patient condition, e.g., the patient's perception of an abnormal condition that differs from normal function, sensation, or appearance, which may include, without limitations, physical disabilities, morbidity, pain, and other changes from the normal condition experienced by a subject.

The terms “drug”, “therapeutic agent”, and the like as used herein refer to a chemical entity or biological product, or combination of chemical entities or biological products, administered to a subject to treat or prevent or control a disease or condition. The drug or therapeutic agent can be formulated with one or more pharmaceutically acceptable carriers. Therapeutic agents of the instant invention can be co-administered with other drugs or therapeutic agents. “Co-administering,” as used herein refers to the administration with another agent, either at the same time, in the same composition, at alternating times, in separate compositions, or combinations thereof.

As used herein, the terms “effective” and “effectiveness” includes both pharmacological effectiveness and physiological safety. Pharmacological effectiveness refers to the ability of the treatment to result in a desired biological effect in the patient. Physiological safety refers to the level of toxicity, or other adverse physiological effects at the cellular, organ and/or organism level (often referred to as side-effects) resulting from administration of the treatment. On the other hand, the term “ineffective” indicates that a treatment does not provide sufficient pharmacological effect to be therapeutically useful, even in the absence of deleterious effects, at least in the unstratified population. (Such a treatment may be ineffective in a subgroup that can be identified by the expression profile or profiles.) “Less effective” means that the treatment results in a therapeutically significant lower level of pharmacological effectiveness and/or a therapeutically greater level of adverse physiological effects, e.g., greater liver toxicity.

Thus, in connection with the administration of a drug, a drug which is “effective against” a disease or condition indicates that administration in a clinically appropriate manner results in a beneficial effect for at least a statistically significant fraction of patients, such as an improvement of symptoms, a cure, a reduction in disease signs or symptoms, extension of life, improvement in quality of life, or other effect generally recognized as positive by medical doctors familiar with treating the particular type of disease or condition.

The term “effective amount” refers to a dosage or amount that is sufficient to reduce, halt, or slow tumor progression to result in alleviation, lessening or amelioration of symptoms in a patient or to achieve a desired biological outcome, e.g., slow or stop tumor growth or reduction or disappearance of a tumor.

“Enriched” as used herein is understood as to process so as to add or increase the proportion of a desirable ingredient. As used herein, enrichment is understood as increasing the proportion of a specific cell type from a population of cells, e.g. peripheral blood or bone marrow, for the presence of a specific cell type, particularly immune cells based on the presence or absence of specific cell surface markers. Enrichment includes cell sorting by methods such as flow cytometery which rely on sorting based on markers. Enriched, as used herein, does not include treating a cell population with a specific agent (e.g., an antibody, a drug) to increase the proliferation of one or more cell types and/or suppress the proliferation of one or more cell types.

“Essentially” as used herein is understood as not departing from the fundamental nature or critical element of the method or agent. For example, a culture that is static is understood as being essentially static, that is growth of the culture occurs without stirring or agitation, however, the culture may be moved during the period of cell culture without altering the fundamental nature of a static culture.

“Expanding” as used herein is understood as promoting the growth or growing, particularly promoting the growth of a particular cell type within a mixed cell population, e.g., promoting the growth of marrow infiltrating lymphocytes in a mixed population of cells such as fractionated bone marrow from which most of the red blood cells and neutrophils have been removed.

As used herein, “hematological malignancy” is any type of cancer that affects blood, bone marrow, and/or lymph nodes. As the three are intimately connected, a disease affecting one of the three will often affect the others as well: although lymphoma is technically a disease of the lymph nodes, it often spreads to the bone marrow, affecting the blood. Hematological malignancies include, but are not limited to multiple myeloma, leukemias, and lymphomas.

As used herein, “isolated” or “purified” when used in reference to a polypeptide means that a naturally polypeptide has been removed from its normal physiological environment (e.g., protein isolated from plasma or tissue) or is synthesized in a non-natural environment (e.g., artificially synthesized in a heterologous system). Thus, an “isolated” or “purified” polypeptide can be in a cell-free solution or placed in a different cellular environment (e.g., expressed in a heterologous cell type). The term “purified” does not imply that the polypeptide or cell is the only polypeptide or cell present, but that it is essentially free (about 80-90%, or about 90-95%, up to 99-100% pure) of cellular or organismal material naturally associated with it, and thus is distinguished from naturally occurring polypeptide. “Isolated” when used in reference to a cell means the cell is in culture (i.e., not in an animal), either cell culture or organ culture, of a primary cell or cell line. Cells can be isolated from a normal animal, a transgenic animal, an animal having spontaneously occurring genetic changes, and/or an animal having a genetic and/or induced disease or condition. Isolated cells can be further modified to include reporter constructs or be treated with various stimuli to modulate expression of a gene of interest. A cell can also be isolated from a specific reagent, for example, agents used to treat the cells such as antibody-coated beads. Isolation of the cells from antibody-coated beads includes removal of a sufficient portion of the beads such that the cells are acceptable for administration to a subject, particularly a human subject.

As used herein, “kits” are understood to contain at least a non-standard laboratory reagents or device component for use in the methods of the invention in appropriate packaging with directions for use. The kit can further include any other components required to practice the method of the invention, as dry powders, concentrated solutions, or ready to use solutions. In some embodiments, the kit comprises one or more containers that contain reagents for use in the methods of the invention; such containers can be boxes, ampules, bottles, vials, tubes, bags, pouches, blister-packs, or other suitable container forms known in the art. Such containers can be made of plastic, glass, laminated paper, metal foil, or other materials suitable for holding reagents.

“Lymphoablation” as used herein is understood as any form of therapy that can include chemotherapy, antibody, or radiation therapy aimed at depleting lymphocytes in vivo but not depleting myeloid elements. Lymphablation can be performed using any of a number of chemotherapy or immunotherapy agents such as those used for the treatment of lymphomas including, but not limited to, adriamycin, bexxar, blenoxane, dacarbazine, cyclophosphamide, cytoxan, DTIC, etoposide, matulane, mechlorethamine, mustargen, Rituxan, VCR, orasone, procarbazine, vincristine, and Zevalin® (radioimmunotherapy regimen), or combinations thereof.

As used herein, a “marrow infiltrating lymphocyte” or “MIL” is understood as a T cell present in the bone marrow, particularly a T cell present in the bone marrow of a subject suffering from a hematological malignancy or a metastatic neoplastic disease in which tumor cells are present in the bone marrow. MILs can be activated to become aMILs by stimulation with appropriate factors such as anti-CD3 and anti-CD28 antibodies. Fold expansion of aMILS is determined by detection of CD3 cells in the population. The percentage of CD3 is determined on the last day of expansion by staining for CD3. This percentage is multiplied by the total number of cells collected. The total number of CD3 on the last day of expansion is divided by the total number of CD3 on D0 and this is the total fold expansion. Roughly between 10-150 fold expansion is typically achieved using the culture methods provided herein.

A “myeloid-derived suppressor cell” or “MDSC” is understood as a cell that suppresses the immune response to specific antigens. MDSCs are characterized by the presence of markers, on the surface including, but not limited to, Gr-1⁺/CD11b⁺ in mice, and CD14+/CD15+/HLA-DRlo/IL4Ra/CD11b/CD33 and IL4Rα in humans. As used herein, an agent, such as a PDE-5 inhibitor “effective blocking MDSC function” is understood as an agent that blocks the suppressive function of MDSC's on T cell proliferation but does not eliminate the population.

The phrase “pharmaceutically acceptable carrier” is art recognized and includes a pharmaceutically acceptable material, composition or vehicle, suitable for administering compounds used in the methods described herein to subjects, e.g., mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

“Phosphodiesterase 5 inhibitor” or “PDE-5 inhibitor” is understood as any is a drug used to block the degradative action of phosphodiesterase type 5 on cyclic GMP in cells. Agents increasing intracellular cGMP levels can induce either positive or negative effects on NOS2 in a cell dependent manner. In macrophages, for example, cGMP analogues inhibit NOS2 expression. Phosphodiesterase-5 (PDE-5) inhibitors such as (sildenafil (Viagra®), vardenafil (Levitra®), tadalafil (Clalis®)) increase intracellular concentrations of cGMP with therapeutic implications that include the treatment of erectile dysfunction, pulmonary hypertension, and cardiac hypertrophy. SLx-2101 made by Surface Logix® is a PDE-5 inhibitor for use in the methods of the invention. Other commercially available PDE-5 inhibitors can be used in the method of the invention including, but not limited to, sildenafil, vardenafil, tadalafil, dasantafil, avanafil, and LAS34179. Chemical names corresponding to the common names include Tadalafil ((6R,12aR)-2,3,6,7,12,12a-Hexahydro-2-methyl-6-(3,4-methylene-dioxyphenyl-)pyrazino(1′,′:1,6) pyrido(3,4-b)indole-1,4-dione), Vardenafil (2-(2-Ethoxy-5-(4-ethylpiperazin-1-yl-1-sulfonyflphenyl)-5-methyl-7-propy-1-3H-imidazo (5,1-f) (1,2,4)triazin-4-one), Sildenafil (3-[2-ethoxy-5-(4-methylpiperazin-1-yl)sulfonyl-phenyl]-7-methyl-9-propyl-2,4,7,8-tetrazabicyclo[4.3.0]nona-3,8,10-trien-5-one), Udenafil 5-[2-propyloxy-5-(1-methyl-2-pyrrolidinylethylamidosulfonyl)phenyl]-methyl-1-3-propyl-1,6-dihydro-7H-pyrazolo(4,3-d)pyrimidine-7-one, Dasantafil 7-(3-Bromo-4-methoxybenzyl)-1-ethyl-8-[[(1,2)-2-hydroxycyclopentyl]amino]-3-(2-hydroxyethyl)-3,7-dihydro-1-purine-2,6-dione, Avanafil 4-{[(3-chloro-4-methoxyphenyl)methyl]amino}-2-[(2S)₂-(hydroxymethyl)-pyrrolidin-1-yl]-N-pyrimidin-2-ylmethyl)pyrimidine-5-carboxamide, LAS 34179 Triazolo[1,2-]xanthine, 6-methyl-4-propyl-2-[2-propoxy-5-(4-methylpiperazino)sulfonyl]phenyl-, Roflumilast (3-(cyclopropylmethoxy)-N-3,5-dichloropyridin-4-yl)-4-difluoromethoxy)ben-zamide), Cilomilast (4-cyano-4-(3 cyclopentoxy-4-methoxy-phenyl)-cyclohexane-1-carboxylic acid), and Piclamilast (3-cyclopentoxy-N-3,5-dichloropyridin-4-yl)-4-methoxy-benzamide). Other PDE-5 inhibitors are provided, for example, in US Patent Publications 20090074796 and 20090186896; and U.S. Pat. No. 6,362,178, which are each incorporated herein by reference in their entirety. By “PDE-5 inhibitor” is meant a compound that inhibits cGMP hydrolysis by phosphodiesterase-5. PDE-5 inhibitors preferably reduce PDE-5 enzymatic activity by at least 5% (e.g., 10%, 15%, 20%, 30%, 50%, 60%, 75%, 85%, 90% or 95%). Methods for assaying the activity of a PDE-5 inhibitor are known in the art and provided, for example, in US Patent Publication 20090074796 and U.S. Pat. No. 6,362,178.

“Providing,” refers to obtaining, by for example, buying or making the, e.g., cells, polypeptide, drug, polynucleotide, probe, and the like. The material provided may be made by any known or later developed biochemical or other technique. Cells can be obtained from a subject to be treated using the methods of the invention.

“Regulatory T cells” or “Tregs” are understood as a specialized subpopulation of T cells that act to suppress activation of the immune system and thereby maintain immune system homeostasis and tolerance to self-antigens in a normal subject. In a subject suffering from neoplastic disease, Tregs can suppress an immune response to the tumor. Regulatory T cells come in many forms, including those that express the CD8 transmembrane glycoprotein (CD8+ T cells), those that express CD4, CD25 and Foxp3, and other T cell types that have suppressive function.

A “sample” as used herein refers to a biological material that is isolated from its environment (e.g., blood or tissue from an animal, cells, or conditioned media from tissue culture) and is suspected of containing, or known to contain an analyte or other desired material. A sample can also be a partially purified fraction of a tissue or bodily fluid, e.g., from a subject having a specific disease or condition. A reference sample can be a “normal” sample, from a donor not having the disease or condition fluid. A reference sample can also be from an untreated donor or cell culture not treated with an active agent (e.g., no treatment or administration of vehicle only) or not subjected to conditions to induce a disease state. A reference sample can also be taken at a “zero time point” prior to contacting the cell with the agent to be tested.

As used herein, “small molecule” is understood to refer to a chemical compound having a molecular weight of 1500 Da or less, 1250 Da or less, 1000 Da or less, 750 Da or less, or 500 Da or less. In certain embodiments, “small molecule” does not include peptide or nucleic acid molecules.

As used herein, a “static culture” is understood as a cell culture that is grown without essentially continuous movement. In a stationary culture, the culture container is preferably placed directly on a non-moving surface, e.g., a shelf or rack in a tissue culture incubator. It is understood that the culture can be moved for example into a tissue culture hood for tissue culture maintenance, testing and analysis, etc. of the culture. A stationary culture is not grown on a rocking or rotating platform and is not a spinner culture.

A “subject” as used herein refers to living organisms. In certain embodiments, the living organism is an animal. In certain preferred embodiments, the subject is a mammal. In certain embodiments, the subject is a domesticated mammal. Examples of subjects include humans, non-human primates, dogs, cats, mice, rats, cows, horses, goats, and sheep. A human subject may also be referred to as a patient. A subject can also be a cadaver.

As used herein, “substantially removed” for example, wherein a specific cell type is substantially removed from a population of cells, is understood is at least 75%, at least 80%, at least 85%, at least 90%, at least 95%, at least 98%, or all detectable cells of a certain type are removed from a population of cells. In a preferred embodiment, cells are subjected to density gradient centrifugation or other methods that do not include labeling of cells for the purpose of sorting one type of cell from anther. However, methods of cell staining and sorting can be used to determine if the desired cell population has been substantially removed from the initial population of cells. In a preferred embodiment of the invention, the bone marrow is subjected to density gradient centrifugation to substantially remove red blood cells and neutrophils.

A subject “suffering from or suspected of suffering from” a specific disease, condition, or syndrome has at least one risk factor and/or presents with at least one sign or symptom of the disease, condition, or syndrome such that a competent individual would diagnose or suspect that the subject was suffering from the disease, condition, or syndrome. Methods for identification of subjects suffering from or suspected of suffering from hematological malignancy or metastatic disease with bone marrow involvement is within the ability of those in the art. Methods of identifying specific genetic or lifestyle predispositions to hematological malignancies is well within the ability of those of skill in the art. Subjects suffering from, and suspected of suffering from, a specific disease, condition, or syndrome are not necessarily two distinct groups.

The language “therapeutically effective amount” or a “therapeutically effective dose” of a compound is the amount necessary to or sufficient to provide a detectable improvement in of at least one symptom associated or caused by the state, disorder or disease being treated. The therapeutically effective amount can be administered as a single dose or in multiple doses over time. Two or more compounds can be used together to provide a “therapeutically effective amount” to provide a detectable improvement wherein the same amount of either compound alone would be insufficient to provide a therapeutically effective amount. “Therapeutically effective amount,” as used herein refers to an amount of an agent which is effective, upon single or multiple dose administration to the cell or subject, decreasing at least one sign or symptom of the disease or disorder, or prolonging the survivability of the patient with such a disease or disorder beyond that expected in the absence of such treatment.

An agent can be administered to a subject, either alone or in combination with one or more therapeutic agents, as a pharmaceutical composition in mixture with conventional excipient, e.g., pharmaceutically acceptable carrier.

The pharmaceutical agents may be conveniently administered in unit dosage form and may be prepared by any of the methods well known in the pharmaceutical arts, e.g., as described in Remington's Pharmaceutical Sciences (Mack Pub. Co., Easton, Pa., 1980). Formulations for parenteral administration may contain as common excipients such as sterile water or saline, polyalkylene glycols such as polyethylene glycol, oils of vegetable origin, hydrogenated naphthalenes and the like. In particular, biocompatible, biodegradable lactide polymer, lactide/glycolide copolymer, or polyoxyethylene-polyoxypropylene copolymers may be useful excipients to control the release of certain agents.

The compounds of the invention can, for example, be administered by injection, preferably intravascularly, that is intravenously. Methods of administration by injection or infusion can be performed using a pump. The methods herein contemplate administration of an effective amount of compound or compound composition to achieve the desired or stated effect. Typically, the pharmaceutical compositions of this invention will be administered once a day, once a week, every two weeks, once a month, or more or less frequently, depending on the specific needs of the subject to be treated. The specific pharmacokinetic and pharmacodynamic properties of the composition (e.g., persistence and engraftment of the MILs) to be administered will effect dosing. Such administration can be used as a chronic or acute therapy. The amount of active ingredient that may be combined with the carrier materials to produce a single dosage form will vary depending upon the host treated and the particular mode of administration. A typical preparation will contain from about 1% to about 95% active compound (w/w). Alternatively, such preparations contain from about 20% to about 80% active compound.

Doses used herein in animal experiments correspond to doses of about 1.7×10⁸/kg, 8.3×10⁷/kg, and 3.3×10⁷/kg of activated MILs. It is understood that any dose within the range of 5×10⁸/kg to about 1×10⁷/kg, or about 1.7×10⁸/kg to about 3.3×10⁷/kg activated aMILs would be useful in the method of the invention. It is further understood that dosages may be higher or lower, and that the specific dosage may be altered based on the amount of cells present, the specific disease to be treated, the severity of the disease to be treated, and other considerations known to those of skill in the art.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained. Patients may, however, require intermittent treatment on a long-term basis upon any recurrence of one or more signs or symptoms of cancer.

Pharmaceutical compositions of this invention comprise compounds of the invention or a pharmaceutically acceptable salt thereof. Further, pharmaceutical compositions of the instant invention can be administered with other pharmaceutical agents used for the treatment of cancer or other hyperplastic disorders.

It will be appreciated that the actual preferred amounts of active compounds used in a given therapy will vary according to e.g., the specific compound being utilized, the particular composition formulated, the mode of administration and characteristics of the subject, e.g., the species, sex, weight, general health and age of the subject. Optimal administration rates for a given protocol of administration can be readily ascertained by those skilled in the art using conventional dosage determination tests conducted with regard to the foregoing guidelines.

The term “therapy” refers to a process that is intended to produce a beneficial change in the condition of a mammal, e.g., a human, often referred to as a patient. A beneficial change can, for example, include one or more of restoration of function, reduction of symptoms, limitation or retardation of progression of a disease, disorder, or condition; or limitation or retardation of deterioration of a patient's condition, disease or disorder.

Cells and/or subjects may be treated and/or contacted with one or more standard cancer therapeutic treatments including, surgery, chemotherapy, radiotherapy, gene therapy, immune therapy, anti-angiogenic therapy, hormonal therapy, bone marrow transplant, or other therapy recommended or proscribed by self or by a health care provider.

“Therapeutically effective amount,” as used herein refers to an amount of an agent which is effective, upon single or multiple dose administration to the cell or subject, in prolonging the survivability of the patient with such a disorder beyond that expected in the absence of such treatment.

Treatment, amelioration, and/or treatment of a disease is practiced on a subject first identified as being prone to or suffering from a disease or condition. During and after treatment and amelioration of a disease or condition, a subject can be monitored for signs or symptoms of the disease or condition.

The dosage of the MILs of the present invention will depend on the disease state or condition being treated and other clinical factors such as weight and condition of the human or animal and the route of administration of the compound. It is to be understood that the present invention has application for both human and veterinary use. The methods of the present invention contemplate single as well as multiple administrations, given either simultaneously or over an extended period of time.

Formulations suitable for parenteral administration include aqueous and non-aqueous sterile injection solutions which may contain anti-oxidants, buffers, bacteriostatics, and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampoules and vials, and may be stored in a freeze-dried (lyophilized) condition requiring only the addition of the sterile liquid carrier, for example, water for injections, immediately prior to use. Methods to prepare the MIL of the invention for administration to a subject, typically by intravascular administration, is well within the ability of those of skill in the art. Extemporaneous injection solutions and suspensions may be prepared from sterile powders, granules and tablets of the kind previously described.

Preferred unit dosage formulations are those containing a daily dose or unit, daily sub-dose, as herein above recited, or an appropriate fraction thereof, of the administered ingredient. It should be understood that in addition to the ingredients, particularly those mentioned above, the formulations of the present invention may include other agents conventional in the art having regard to the type of formulation in question.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

“At least” a particular value is understood to mean that value or more. For example, “at least 2” is understood to be the same as “2 or more” i.e., 2, 3, 4, 5, 6, 7, 8, 9, 10, 11, 12, 13, 14, 15, etc.

Unless specifically stated or obvious from context, as used herein, the term “or” is understood to be inclusive.

Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All references, patents, patent applications, and Accession Numbers as of the filing date of the priority application referred to herein are specifically incorporated by reference.

### DETAILED DESCRIPTION OF THE INVENTION

The invention provides methods of treatment of subjects with cancer, particularly hematological malignancies, such as leukemias and lymphomas, by providing activated marrow infiltrating lymphocytes (MILs) to the subject. The activated MILs can be prepared, for example, using the devices and methods provided herein.

The invention provides devices and methods for the preparation of activated marrow infiltrating lymphocytes. In certain embodiments, the preparation methods include treatment of the cells with a PDE-5 inhibitor.

In patients with any of a number of types of cancer, particularly hematologic malignancies, T cells can easily be obtained from the bone marrow microenvironment with heightened tumor specificity as compared to peripheral blood. By comparing T cells obtained from these two different compartments from a subject having a hematological malignancy, we have demonstrated oligoclonal restriction of marrow infiltrating lymphocytes (MILs) obtained from marrow aspirates. Anti-CD3/CD28 antibody-conjugated magnetic beads were used to stimulate the bone marrow cells in vitro for 7 days to generate activated MILs. The activated MILs show a greater expansion and enhanced tumor activity as compared to peripheral blood lymphocytes in all patients examined. Without wishing to be bound by mechanism, these findings suggest that: 1) the marrow is a reservoir of tumor-specific T cells; 2) MILs can be activated and expanded in all patients studied (as compared to the limited numbers observed in metastatic melanoma); 3) these cells traffic to the bone marrow upon infusion; 4) persist for up to 200 days following adoptive transfer in NOD/SCID mice; and that 5) activated MILs are capable of eradicating pre-established disease and targeting myeloma stem cell precursors thus implying a broad antigenic recognition.

The treatment strategy provided herein can significantly enhance the efficacy of adoptive immunotherapy through the expansion of this tumor-specific MILs cell population with a greater antigenic specificity compared to peripheral blood lymphocytes (PBLs) stimulated in a similar manner. The ability to infuse these autologous polyclonal tumor specific T cells and ultimately integrate the adoptive transfer of these cells with tumor-specific vaccinations or other approaches to augment T cell activity such as, but not limited to, CTLA-4 or PD-1 blockade are the goals of the clinical implementations of such an approach.

Provided herein are methods for generating sufficient activated MILs for treatment of large mammals, e.g., humans, under conditions that the activated MILs would be acceptable for administration to humans. The invention provides a device, specifically a culturing bag and support, to allow for generation of sufficient quantities of cells for treatment.

Adoptive immunotherapy is gaining importance as a therapeutic option in the treatment of many malignancies. In hematologic malignancies, the clinical benefits of donor lymphocyte infusions (DLI) administered in the context of an allogeneic stem cell transplant must weigh the limited HLA-compatibility and significant morbidity and mortality associated with this intervention with the clinical benefit. A major concern of such an approach is the lack of the T cell tumor specificity and the broad non-specific T cell recognition resulting in graft vs. host disease.

To be successful, adoptive immunotherapy must fulfill several basic criteria: 1) T cells must be present with a pre-existing tumor specificity; 2) the T cells need to be activated and expanded to sufficient numbers to effectively impart an anti-tumor effect; 3) T cells must possess broad antigen recognition; 4) T cells must be able to traffic to the tumor site; 5) they must recognize and kill the tumor upon encounter; 6) they must persist over time following their infusion.

MILs possess all of these critical criteria. Tumor specific T cells can be effectively expanded from the bone marrow in all patients examined Upon activation, MILs possess antigenic specificity that recognizes both mature plasma cells as well as the myeloma stem cell precursors. Their expression of the surface chemokine, CXCR4, facilitates their trafficking to the bone marrow upon reinfusion. Furthermore, in a NOD/SCID murine model of adoptive transfer, we observed persistence of activated MILs up to 210 days post-infusion in the marrow of these mice (and no evidence of myeloma) whereas activated PBLs exerted no measurable anti-tumor effect and T cells were never seen in the marrow.

Lytic bone disease induced by osteoclast activation is a major complication in multiple myeloma. This usually results from plasma cell-induced activation of osteoclasts through the production of RANK/L and MIP1α. Data from our laboratory suggests that resident MILs likely also contribute to osteoclast activation. Without wishing to be bound by mechanism, it is suggested that the tumor microenvironment through its production of IL-6 induces a Th17 phenotype of the MILs which is responsible for increased osteoclast activation. Interestingly, activation of MILs with anti-CD3/CD28 antibody-conjugated magnetic beads shifts the phenotype of the MILs from Th17 (IL-17 producing) to Th1 (γIFN producing). It is suggested that the production of γIFN can significantly abrogate osteoclast activation. Taken together, our data demonstrates a role of the marrow-residing MILs in mediating the destructive bone lesions in myeloma. More importantly, however, we have also shown how in addition to exerting a significant anti-tumor effect towards the plasma cells and their precursors, activated MILs significantly reduce osteoclast formation and may likely play a significantly role in reducing the lytic disease associated with the disease.

The invention provides devices and methods to expand T cells, preferably in the context of bone marrow, with heightened tumor specificity from the bone marrow microenvironment in a polyclonal but highly tumor-specific manner. This is a process wherein the marrow microenvironment is used to sustain and expand T cells. There is roughly a 75 to 100-fold expansion of MILs in a 7-day expansion process, with potentially greater expansion in the presence of PDE-5 inhibitors. In contrast to many currently used adoptive T cell therapeutic approaches, the T cell manufacturing process provided herein requires no specific intervention to select for tumor specificity and does require the presence of tumor in the marrow at the time of T cell expansion. Using the methods provided herein, the T-cells, which represent a minority of the total bone marrow cell population are expanded in the presence of almost complete bone marrow.

To assure maximal tumor—T cell contact, the aspirated bone marrow is fractionated on a Lymphocyte Separation Medium density gradient and cells are collected almost to the level of the red cell pellet. This separation method removes substantially only the red blood cells and the neutrophils, providing nearly complete bone marrow, and results in the collection of both T cells as well as tumor cells. T-cells are expanded without a T-cell specific separation step, and without a tumor cell separation step. Cell type specific separation steps include, for example, cell labeling using antibodies or other cell-type specific detectable labels, and sorting using fluorescence activated cell sorting (FACS). The methods of the invention can be practiced without such labeling and cell sorting methods.

We have also determined that it is critical to maximize the bead-T cell contact during the first 24-48 hours of culture. As the T-cells represent only a minority of the total cells in the population, contact of the T-cells with the antibody coated beads is promoted by the use of a sufficient number of beads to cells, in the range of about 1:1 to about 5:1 beads to cells, preferably about 2:1 to 4:1 beads to cells, preferably about 2.5:1 to 3.5:1 beads to cells. It is understood that the ratios provided are for the beads provided, and that a change in the size of the beads and/or the density of antibodies on the beads can alter the bead:cell ratio. Further, we created a device for culturing the cells. The device provides a smooth, rigid, rounded bottom surface to promote collection of the cells and beads by gravity in close proximity. The device includes an enclosed cell container (further described herein) that rests on a support. During at least the first 3 days of culture in the presence of the beads, the container is stationary (i.e., no rocking or rotation) to further promote contact between the beads and the cells. These steps and conditions are essential to maximizing the expansion of tumor-specific MILs to allow for the production of sufficient cells to be therapeutically useful. Further, the culture conditions promote growth of the T cells without promoting growth of the tumor cells. This is an essential element of the invention.

A preferred device for culturing the cells for use in the methods of the invention is provided by the invention.

The invention provides a device for expanding MILs for use in the methods of the invention. The device is preferably manufactured to meet the requirements of the Food and Drug Administration or other local drug regulatory agency. However, it is understood that the device can be used for laboratory and other research applications such that the device need not be manufactured to such rigorous standards. However, the device should be made using gas (e.g., O₂ and CO₂) permeable materials that can be sterilized, for example using gamma radiation. Further, the device should be a closed culture system, and all inlets and outlets of the device are selected and manufactured to allow for culturing cells under conditions acceptable to the FDA for subsequent administration of the cells to a human subject. Such conditions are well known to those of skill in the art.

The device 1 of the invention includes an enclosed cell container 3 that includes a smooth, rigid rounded bottom surface 5. The cell container bottom surface can be rigid as a result of an inherent property of the material that it is made out of (e.g., rigid plastic). Alternatively, the cell container bottom surface can be rigid as a result of inflation of a bag or other flexible material with a sufficient amount of air to make the bag firm to the touch and provide a smooth, rigid, rounded bottom surface. The overall shape of the cell container can be essentially any shape. In the embodiment shown, the cell container is nearly circular. In other embodiments, the cell container can be elliptical, oval, or any other shape including a rounded bottom surface, e.g., the top of the device need not be rounded.

In a preferred embodiment, the area of the rigid bottom surface should be sufficient to promote contact of the cells with the beads, particularly during the static phase of the culture. Appropriate volumes can be determined by one of skill in the art. Further, the rigid portion of the container should extend high enough such that the volume of the cells in media 11 should not be higher than the rigid portion of the container.

In certain embodiments where the cell container is made of a flexible material to be inflated with air, the container may include a seam that runs horizontally around the bag, preferably at about the center of the bag. The seam is preferably designed to prevent collection of cells, beads, or other materials at the seam. Alternatively, culture volumes can be selected such that the combined volume of the materials 11 in the cell container (e.g., cells, growth media, beads) does not reach the level of the seam.

The specific volume cultured in the cell container will vary depending on the amount of starting material, the day in culture (i.e., as the culture expands, the volume of cells and media expands), the size of the subject to be treated (e.g., pediatric cancers will likely be for smaller subjects requiring smaller numbers of cells). Typically, commercially available culture bags are provided having different volumes for culturing different volumes of cells. These commercially provided guidelines can be used to select an appropriate culture volume. Typically, cells for use in the methods of the invention will be grown in a volume of at least 40 ml, 50 ml, 60 ml, 70 ml, 80 ml, 90 ml, 100 ml, 110 ml, 120 ml, 130 ml, 140 ml, 150 ml, 160 ml, 170 ml, 180 ml, or 190 ml. Typically, cells for use in the methods of the invention will be grown in a volume of no more than 200 ml, 225 ml, 250 ml, 275 ml, 300 ml, 350 ml, 400 ml, 450 ml, 500 ml, 1000 ml, 1250 ml, 1500 ml, or 2000 ml; or within any range that is provided by the upper and lower limits provided. For example, commercially available bags (e.g., Nexell 1000 ml Lifecell Tissue Culture Flask) have a flat (i.e., deflated) surface area of 300 cm² (17 cm×20 cm) and a fill volume of about 100-500 ml. Such containers are typically used for the initial culture of the invention having a volume of about 40 ml.

The cell container 3 contains at least a first port 7 and a second port 7′ to allow for material to be put into the cell container, and the release of air and other materials from the cell container. It is understood that the cell container can include any number of ports as long as they do not interfere with the shape of the rounded bottom surface 5 of the cell container 3. In certain embodiments, the ports are airtight until disrupted to allow the container to be airtight. In certain embodiments, the ports include fittings or connectors such as luer locks or snap connectors to allow the ports to be coupled to syringes, stopcocks, tubing, or other devices. In certain embodiments, the ports include valves that permit flow in only a single direction. In certain embodiments, the cell container includes a variety of types of ports. The specific number and location of ports is not a limitation of the device. Port types and sizes can be selected to allow for the use of the device of the invention with commercially available products designed for use in bag culture methods.

An appropriately dimensioned support 9 is provided to stabilize the cell container during the activation and culturing steps. The contact surface 13 of the support with the cell container is round and allows for the rounded cell container to be retained in a fixed position with the support resting on a flat surface without the shape of the rigid bottom surface of the cell container being distorted by contact with the surface on which the support rests. In certain embodiments, the contact surface of the support with the container is a recess appropriately dimensioned for contacting and stabilizing the cell container. In certain embodiments, the contact surface of the support with the container is a ring. In certain embodiments, the contact surface of the support with the container is a number of finger-like projections. In certain embodiments, the contact surface of the support with the container is any of a combination of the above named structures. The invention is not limited by the specific shape of the support and the contact surface with the container. The support is limited only in that it must not distort smooth rounded shape of the culture container, and it must be able to retain the culture container in a fixed position. In certain embodiments, the support can include adhesives, clips, fasteners, or other devices to further secure the cell container on the support. In certain embodiments, the support can be used in conjunction with a rocker, a rotating platform, stir plate, or other device for culturing the cells with movement. In certain embodiments, the support can be designed to allow for stacking of culture devices of the invention.

The drawings show the fill level of the cells and media in the cell container being higher than the top of the contact surface between the cell container and the support. In a preferred embodiment, the fill level of the cells and media is below the top of the contact surface between the cell container and the support. Having a smaller fill volume can promote a smooth surface of the bottom of the cell culture container.

In certain embodiments, the invention provides multiple linked culture containers to allow for portioning of cells into separate culture spaces during expansion.

The invention provides kits for use in the culture methods of the invention. The kits include a culture bag for culturing of mammalian cells (e.g. LIFECELL Tissue Culture Flasks available for example from Kal Medical Supplies, Inc) and an appropriately dimensioned support to stabilize the rigidly inflated cell culture bag during the activation and culturing steps. The support is round and allows for the rounded cell container to rest stably on a flat surface without the shape of the rigid bottom surface of the cell container being distorted by contact with the surface on which the support rests. In certain embodiments, the kits can include more than one support to allow for optimization of the smooth, rigid bottom surface of the cell container when an inflated cell container is to be used. For example, when using larger volumes of media, the weight of the fluid in the cell container could result in distortion of the smooth surface of the base of the cell container.

Resident antigen presenting cells are capable of antigen uptake, processing, and presentation to the MILs. Should this yield similar tumor specificity, this would enable us to expand tumor specific in patients with hematologic malignancies in complete remission or with solid tumors with minimal bone marrow involvement at the time of the bone marrow harvest. Furthermore, in attempting to further increase the overall expansion and tumor-specificity of activated MILs, we have recently observed that the addition of PDE-5 inhibitors to the tissue culture medium can increase the MILs fold expansion 3-4 fold. Without being bound by mechanism, this is likely occurring through the abrogation of NO and arginase-1 production generated by myeloid-derived suppressor cells (MDSCs).

A critical issue regarding the generation of tumor-specific T cells for adoptive therapy is the ability to determine their degree of tumor specificity. We have generated an in vitro read-out whereby the T cells are co-cultured with either autologous tumor, HLA-restricted allogeneic tumor cell lines, or cell lysate and the degree of tumor specificity is determined after a pre-determined period of anti-CD3/CD28 activation by ³H-thymidine incorporation or CFSE dilution of the MILs upon co-culture with tumor. A major difference between activated MILs and PBLs is our ability to demonstrate significant anti-tumor efficacy of MILs upon activation. The in vitro assay enabled us to determine the maximal time-frame for MILs expansion.

Lastly, our findings that the residing MILs in myeloma possess a paucity of Tregs and a reciprocal predominance of Th17 T cells has significant biologic as well as clinical implications. The absence of Tregs likely augments the overall tumor specificity of our activation/expansion procedure provided herein. Furthermore, it is highly likely that upon infusion into patients there will be less in vivo expansion of Tregs and thus further potentiation of the effector arm of the T cell response. Furthermore, our findings suggesting that Th17 cells play a critical role in the osteoclast-induced bone disease and that their activation converts the MILs into Th1 γIFN producing cells raises the possibility of utilizing adoptive T cell therapy with MILs to impact the progression of lytic bone disease in myeloma.

The activation and expansion of MILs and the demonstration of their greater antitumor specificity towards both mature plasma cells as well as their clonogenic precursors as compared with their peripheral blood counterparts is demonstrated. Importantly, several attributes of MILs make them suitable candidates for immunotherapy. Specifically, under the conditions described herein, they expand more rapidly upon stimulation than PBLs and often maintain a skewed T-cell repertoire upon activation, possibly suggesting augmented tumor specificity. Whereas the unactivated MILs show profound hyporesponsiveness toward autologous tumor, the ability to activate and expand T cells and markedly enhance their tumor reactivity argues against deletional tolerance as a presumptive mechanism mediating T-cell unresponsiveness in this setting. Furthermore, activated MILs show tumor specificity with little cross-reactivity towards nonmalignant hematopoietic elements, have a higher expression of CXCR-4, and possess a greater responsiveness to SDF-1, suggesting an increased migratory ability of MILs to the bone marrow. Taken together, these findings show the ability to activate and expand marrow-infiltrating T cells with a memory/effector phenotype that seem to target the broad range of tumor antigens present on both mature terminally differentiated plasma cells as well as their precursors and possess chemokine receptors that would seem to facilitate trafficking to the bone marrow compartment—features that would be necessary for maximizing antitumor immunity of adoptive immunotherapy.

In recent years, adoptive immunotherapy has shown measurable clinical activity yet many obstacles still exist to maximize its efficacy. For adoptive immunotherapy to be effective, T cells must overcome the intrinsic tolerogenic mechanisms that often limit immune responsiveness. Specifically they must be tumor-specific, capable of being expanded to clinically meaningful numbers, traffic to the tumor microenvironment upon infusion into the host, and kill the tumor upon its encounter. To date, multiple strategies have been attempted to increase the tumor specificity of adoptive immunotherapy. Whereas antigen-specific T cell lines or T cell clones have been used, they require the need to identify and isolate peptides or tumor antigens thereby limiting this approach to a finite number of known tumor antigens and increasing the likelihood of recurrent disease associated with antigen-escape variants.

Effective adoptive immunotherapy requires activated T cells with broad tumor specificity present in sufficient numbers to achieve a clinically measurable antitumor response. Such qualitative and quantitative requirements have been difficult to realize with many techniques employed to date. The development of the antibody-coated bead-based platform consisting of anti-CD3/anti-CD28 antibodies coupled to magnetic beads has enabled activation and 300- to 500-fold expansion of T cells in a polyclonal manner has been reported using PBLs. Such technology addresses two major requirements for effective immunotherapy, (a) the ability to obtain adequate cell numbers and (b) the activation of lymphocytes, to overcome the unresponsiveness associated with tumor-induced tolerance. Its clinical use has thus far been limited to PBLs. In an effort to increase the tumor specificity of adoptive immunotherapy, we obtained and expanded lymphocytes residing within the bone marrow of multiple myeloma patients. As T lymphocytes are present in the bone marrow of essentially all types of cancer, including non-hematological cancers, the methods provided herein should be effective in treating essentially all types of cancer.

Activation and expansion of MILs was based on two previously reported phenomena: the enhanced tumor specificity of tumor-infiltrating lymphocytes (Rosenberg et al. Science 1986; 233:1318-21) and the demonstration of tumor-reactive T cells in the bone marrow of patients with melanoma (Letsch et al. Cancer Res 2003; 63:5582-6), breast cancer (Feuerer et al. Nat Med 2001; 7:452-8), and multiple myeloma—a disease in which the bone marrow also represents the tumor microenvironment (Dhodapkar et al. Proc Natl Acad Sci USA 2002; 99:13009-13). These previous studies describe the presence of tumor-specific cells in the bone marrow and possibly address their enhanced tumor reactivity as compared with peripheral lymphocytes. It is expected that many other tumor types, particularly metastatic tumors would include tumor-reactive T cells in the bone marrow.

The ability to activate and expand MILs as a means of overcoming their unresponsiveness and significantly increasing their tumor specificity compared with activated PBLs is provided herein. Our experiments show that the presence of tumor in the bone marrow may play a critical role in preserving the antigen specificity of activated MILs. Several hypotheses may explain the increased reactivity of activated MILs over activated PBLs. Without being bound by mechanism, it is suggested that the persistence of antigen in the bone marrow may be essential for the maintenance of a memory response. Anti-CD3/CD28 antibody-coated bead activation may be reversing tolerance in the bone marrow T-cell population. As demonstrated herein, the tumor specificity of activated MILs was dependent on the presence of antigen during T-cell activation. Further, the bone marrow is a functional lymphoid organ capable of mounting both a primary immune response and a secondary responses via reactive lymphoid follicles in the presence of danger signals (infection, inflammation, autoimmunity, and cancer).

T cells in myeloma patients show considerable skewing of the Vβ T-cell receptor repertoire. Such skewing suggests either the selective outgrowth of T cells with marked tumor specificity or results from the profound underlying T-cell defects characteristic of patients with a significant tumor burden. In the latter case, a benefit of polyclonal stimulation of PBLs with the anti-CD3/CD28 antibody-conjugated magnetic beads is the ability to restore a normal T-cell repertoire and thus correct any underlying T-cell defects. In contrast, if the oligoclonal expression of specific Vβ families reflects the presence of T cells with tumor specificity, activation and expansion of this pool of T cells with maintained antitumor activity and T-cell receptor repertoire skewing may be preferable. As demonstrated herein, PBLs normalized their Vβ T-cell repertoire upon activation and expansion with anti-CD3/CD28 antibody-conjugated magnetic beads, whereas MILs maintained the Vβ restriction. Considering the enhanced tumor-specific response of activated MILs, their skewed T-cell repertoire may be suggestive of greater tumor recognition. Without being bound by mechanism, it may be important to conserve and possibly increase the degree of Vβ skewing during T-cell expansion.

As demonstrated herein, the activation and expansion of MILs with anti-CD3/CD28 antibody-conjugated magnetic beads generates potent antitumor activity and the persistence of antigen during this expansion may be of significant importance in maintaining (and augmenting) the tumor specificity. Dhodapkar et al. (2002) have also studied the role of MILs in myeloma patients Similar to our findings, freshly isolated MILs or PBLs showed no activity upon stimulation with autologous tumor or tumor peptides. However, whereas that study saw no significant differences between T cells obtained from the peripheral blood and the marrow compartment in the enzyme-linked immunospot assay following 12 to 16 days of incubation with tumor-pulsed dendritic cells, a 10-fold greater antitumor response of activated MILs over activated PBLs was observed in our system in all assays examined. These discrepant results may be related to potency of anti-CD3/CD28 bead stimulation as compared with dendritic cell activation of MILs. Without being bound by mechanism, what seems to be an increase in frequency of tumor-reactive T cells in the activated and expanded MILs cultures may reflect the breaking of tolerance and restoration of function of tumor-reactive T cells. Furthermore, our stimulation of MILs within the bone marrow microenvironment is another important factor that may explain these results.

Another mechanism that may account for the increased immune responsiveness in the bone marrow could be related to the preferential trafficking of tumor-specific T cells to the bone marrow. Activated MILs exhibited far greater transwell migration in response to SDF-1 than did activated PBLs. Whereas this chemokine is a known bone marrow chemoattractant in addition to being the ligand for CXCR-4 expressed on T cells, it has also been implicated in the migration of myeloma cells to the bone marrow and the establishment of bone marrow metastases of other tumors. It is reasonable to hypothesize that this chemokine-mediated physical colocalization of tumor and T cells to the same compartment may be yet another mechanism by which the bone marrow microenvironment is enriched in the frequency of antigen-specific T cells.

A major requirement for clinically meaningful T-cell immunotherapy is the generation of T cells recognizing a broad spectrum of tumor-specific antigens that possess a measurable effector function. Multiple myeloma represents a disease in which the malignant plasma cell likely represents a terminally differentiated phenotype and not the clonogenic “stem cell.” An increasing body of literature has focused on targeting the cancer “stem cell” as a therapeutic intervention aimed at imparting a sustainable antitumor effect by eliminating the self-renewing source of tumor cells. As such, any therapy that produces long-term remissions is dependent on its ability to inhibit both the terminally differentiated and self-renewing cell populations. Activated MILs show significant anti-myeloma activity against both the terminally differentiated CD138⁺ plasma cells and their clonogenic precursors without affecting normal hematopoietic function. This suggests that activated MILs may target a broad range of tumor-specific antigens in a tumor-specific manner, potentially using both cytotoxic CD4⁺ and CD8⁺ cells, or possibly a small population of exceptionally specific and potent CD8⁺ CTLs. The depth of tumor recognition coupled to the absence of non-tumor-specific activity provides a safe and effective therapeutic approach for multiple myeloma.

In summary, these findings confirm the presence of tumor-specific memory T cells within the marrow that show enhanced antitumor efficacy upon activation and seem to possess features that enhance their trafficking to the bone marrow compartment (also the tumor microenvironment). Such features would likely augment the efficacy of adoptive immunotherapy and provide the rationale for using activated MILs in a therapeutic setting. Furthermore, we describe a device and method for the simple and rapid expansion of T cells with specificity towards a broad range of antigens present not only on the parental tumor but also on its precursor with minimal reactivity towards normal hematopoietic elements using clinically available technology, and highlight several critical requirement for maintaining tumor specificity during T-cell expansion. The clinical implementation of this approach in the proper setting alone or in combination with approaches to further augment tumor specificity such as tumor vaccination may improve our understanding of immunotherapy and increase its therapeutic efficacy.

Further provided herein is the demonstration that PDE-5 blockade exerts an indirect, immune-mediated, antineoplastic effect through inhibition of MDSC-mediated immunosuppressive mechanisms when administered systemically. These findings establish a role for PDE-5 inhibition as a viable and effective immunological adjunct in the treatment of various malignancies, thereby adding to the growing list of therapeutic applications of these agents. Herein we show that PDE-5 inhibition can significantly overcome the immunosuppressive mechanisms present in a tumor-bearing host to generate immune responses that are similar to (if not greater than) those observed in a non-tumor-bearing, vaccine-primed host. However, although a CTL response can be elicited, PDE-5 blockade is incapable of complete tumor eradication. Further provided herein is the use of a PDE-5 inhibitor during the expansion of MILs in the presence of anti-CD3 and anti-CD28 antibodies to increase the expansion of MILs relative to a control culture not including a PDE-5 inhibitor, particularly cultures including a relatively high level of MDSCs.

PDE-5 blockade not only increases intratumoral CD8⁺ infiltration and activation but also enhances their tumoricidal activity. The ability to favorably alter the intratumoral microenvironment, thereby permitting tumor-specific T cells to directly interact with their targets, is critical for maximal antitumor immunity. Effective immunotherapy requires tumor-specific CTLs to infiltrate the tumor and kill their target in situ. Although no clinical advantage was derived by the presence of tumor-specific CTLs in the peripheral blood in melanoma patients, their frequency in the tumor correlated with a favorable prognosis. These clinical observations seem to be confirmed in our model where in vivo sildenafil treatment (a) increased the CD8+ T cell tumor infiltration and (b) increased the percentage of activated T cells. Moreover, the number of tumor-infiltrating T cells directly correlated with a measurable antitumor effect.

Although sildenafil, a PDE-5 inhibitor, can increase cGMP in DCs, and CD11b⁺ cells, the data provided herein indicate that Gr-1⁺/CD11b⁺ MDSCs are its primary cellular target. Gr-1 depletion does not augment sildenafil-mediated antitumor activity, and sildenafil down-regulates MDSC suppressive pathways in vivo. Moreover, sildenafil reverses MDSC suppression in vitro. MDSCs and/or tumor-associated macrophages have been shown to induce apoptosis or anergy in CD8⁺ and CD4⁺ T cells through NOS2- and/or ARG1-dependent mechanisms. In fact, NO production anergizes Th1 cells through inhibition of IL-2 signaling. Alternatively, in a mixed Th1/Th2 cell environment where ARG-induced pathways also mediate immunosuppression, MDSCs produce NO and super-oxide radicals to generate peroxynitrites that induce apoptosis of activated CD8⁺ T cells.

It was previously shown that nitro aspirin, a nitric oxide inhibitor, could abrogate the inhibitory activity of MDSCs by enhancing the preventive and therapeutic efficacy of antitumor vaccines. However, despite its use as a vaccine adjuvant, nitroaspirin demonstrated no antitumor efficacy when used alone. In contrast, down-modulation of both ARG1 and NOS2 in MDSCs with PDE-5 inhibitors effectively abrogates MDSC-mediated immune suppression, resulting in a measurable antitumor response. We previously demonstrated that to effectively exert their suppressive function, MDSCs must (a) be activated by IFN-γ production from antigen-stimulated T cells, (b) release their own IFN-γ, and (c) be responsive to IL-13. Cooperation between these two cytokines leads to the activation of ARG1 and NOS2 enzymes. Sildenafil neither alters IFN-γ production from activated lymphocytes nor changes IL-13 and IFN-γ production from MDSCs. Rather, PDE-5 inhibition down-regulates IL-4Rα expression on MDSCs, likely impairing their responsiveness to IL-13.

MDSCs can promote tumor growth not only by preventing tumoricidal CTL activity but also by supporting tumor angiogenesis. The Gr-1⁺/CD11b⁺ cells, in fact, produce high levels of the matrix metalloprotease 9, which regulates the bioavailability of vascular endothelial growth factor. The selective deletion of the matrix metalloprotease 9 gene in these cells abolishes their ability to promote tumor growth and inhibits tumor formation, likely by limiting migration and/basement membrane degradation.

It is demonstrated herein that PDE inhibitors reduce NOS2 and ARG1. Without being bound by mechanism, it is suggested that PDE-5 inhibitors effect NOS2 mRNA stability. PDE-5 inhibitors destabilize NOS2 mRNA by reducing the ubiquitous mRNA binding protein, human antigen R. As such, destabilization of NOS2 mRNA via PDE-5 inhibition would abrogate NO-mediated immunosuppression more effectively than would competitive inhibition of NO itself. However, because ARG1 mRNA does not possess adenylate/uridylate-rich elements and has not been described to be stabilized by human antigen R, other mechanisms are likely involved in PDE-5-mediated down-regulation of ARG1. One possibility is that high levels of cGMP induced by PDE-5 blockade reduce the cytosolic Ca2+ concentration, leading to a reduction of the calcium-dependent protein kinase C activity that in turn prevents up-regulation of IL-4Rα. The link between IL-4Rα and ARG1 in MDSCs is supported by recent data demonstrating a direct correlation between ARG1 expression and IL-4Rα expression. ACT of tumor-primed CD8 T cells completely eradicated a pre-established C26GM tumor in the LysMCreIL-4Rα-/flox mice in which IL-4Rα expression has been deleted in neutrophils and macrophages, whereas no effect was seen in the control littermates. Our findings support these data by demonstrating that PDE-5 blockade down-regulates IL-4Rα expression on tumor-infiltrating MDSCs and synergizes with the adoptive transfer of tumor-primed CD8+ T cells. This effect appears to specifically target MDSCs, because IL-4Rα expression on isolated CD11b+ cells from tumor-bearing mice is significantly reduced when co-cultured in the presence of sildenafil. Collectively, these findings underscore the critical role of the IL-4Rα-ARG1 pathway in MDSCs, as well as the use of PDE-5 inhibitors as therapeutically effective drugs to overcome tumor-induced immunosuppression.

Although most of the work to date on MDSCs has focused on mouse Gr-1⁺/CD11b⁺ cells, emerging data confirm the presence of these cells in human malignancies. A nonlymphoid CD14⁺ population mediates the hypo-responsiveness in PBLs from MM patients. Although the low proliferative capacity may be caused by intrinsic T cell defects, an additional explanation for T cell unresponsiveness is the presence of a nonlymphoid suppressor population whose function is abrogated by PDE-5 inhibition. Evidence supporting this is the ability of sildenafil to augment the proliferative index of lymphocytes obtained from unfractionated PBMCs, but not from purified CD4⁺ or CD8⁺ T cells. The ability of sildenafil to restore proliferation of PBLs from both head and neck and myeloma patients suggests that the mechanisms found in mice also play a major role in cancer-mediated immunosuppression in two very different human malignancies.

NOS and arginase2 inhibitors such 1-NMMA, Nor-NOHA, NO-aspirin, or Vitamin D3, have all been used in vitro and in mouse models to alter MDSC suppressive mechanisms. Unfortunately, they have either not been extensively tested in humans or have been found to be extremely toxic, as is the case with 1-NMMA. In demonstrating the ability to use clinically available PDE-5 inhibitors to overcome the MDSC-mediated immunosuppressive pathways, these observations open the opportunity for the rapid translation of our preclinical findings into the clinic and give new hope for the development of more effective immune-based treatments for a broad range of human malignancies.

Optionally, activated MILs may be administered in combination with any other standard anti-neoplasia therapy; such methods are known to the skilled artisan and described, for example, in Remington's Pharmaceutical Sciences by E. W. Martin.

### Kits

The invention provides kits for the culturing, activation, and/or administration of the MILs of the invention. In one embodiment, the kit includes one or more non-standard laboratory reagents or devices and instructions for use in appropriate packaging, optionally with instructions for use. Such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

The invention provides kits including at least one non-standard laboratory component for use with the device for culturing the cells of the invention in appropriate packaging, optionally with directions for use. In an embodiment, the invention can include a support for use with the cell culture container of the invention, which may or may not be included in the kit. The invention may further provide tubing, syringes, culture reagents, antibody coated beads, devices and reagents for removing the beads from culture, microscope slides, reagents for staining and/or characterization of cells.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

This invention is further illustrated by the following examples, which should not be construed as limiting.

### EXAMPLES

### Example 1

### Materials and Methods

Methods were performed using methods known to those of skill in the art and as provided in Noonan et al., 2005. Cancer Res. 65:2026-2034 which is incorporated herein by reference.

T-Cell Expansion. Bone marrow and peripheral blood samples were obtained from myeloma patients after having obtained informed consent using an Institutional Review Board-approved protocol. T-cell stimulation was performed by adding anti-CD3/CD28 antibody-coated beads (Xcyte Dynabeads, Xcyte Therapies, Seattle, Wash.) to Ficolled bone marrow or peripheral blood suspended in serum-free conditions at 1×10⁶ cells/mL in AIM-V (Invitrogen, Gibco, Carlsbad, Calif.), 200 μL/well, at a 3:1 bead to T cell ratio. The cells were cultured for 5 days in a 96-well round-bottomed plate at 37° C. with 5% CO₂. The beads were removed from the culture using a magnet. The cells were then replated at 200 μL/well in a 96-well plate for 2 days at 37° C., 5% CO₂. Prior to phenotypic and functional analysis, fluorescence-activated cell sorting (FACS Calibur, BD Biosciences, San Diego, Calif.) analysis was done on bone marrow and peripheral blood by staining for CD3, CD4, CD8, CD25, CD45RA, CD45RO, and CD56 (BD Biosciences, PharMingen, San Diego Calif.). Cell Quest software was used to analyze the results. CD33 and CD138 Selection. Frozen Ficolled bone marrow was thawed and washed thrice in HBSS. The cells were then incubated with either anti-CD138 or anti-CD33 microbeads (Miltenyi Biotec, Auburn, Calif.) for 15 minutes at 4° C. to 6° C. The VarioMACS (Miltenyi) was used to isolate the cells as per protocol of the manufacturer. Proliferation Assays. Media alone, CD138⁺ plasma cells, or CD33⁺ myeloid cells were incubated with either activated or unactivated MILs or PBLs at a 1:1 ratio. Cells were plated at 1×10⁵ CD3⁺ cells/well (+/− stimulus) and incubated for 72 hours in a 96-well plate. They were then pulsed with 1 μCi of [³H]thymidine. Cells were harvested 18 hours later with a Packard Micromate cell harvester. [³H]Thymidine incorporation was measured as counts per minute (cpm) on a Packard Matrix 96 direct β-counter. Values are displayed as the mean±SE cpm.

T-Cell Receptor Spectratyping. RNA was extracted from 5 million cells using Trizol (Invitrogen, Carlsbad, Calif.) and cDNA was prepared using the GeneAmp Gold RNA PCR Reagent Kit (Perkin-Elmer, Wellesley, Mass.). Spectratyping samples were prepared for each Vβ family using a Vβ-specific primer and a common HEX-labeled Cβ primer. The reaction components were 1× AmpliTaq Gold PCR buffer, 1.5 mmol/L MgCl₂, 0.2 mmol/L deoxynucleotide triphosphates, 0.5 μmol/L Cβ-Hex primer, 0.5 μmol/L Vβ-specific primer, 1.25 units AmpliTaq Gold, and 1 μL cDNA template. The thermocycler conditions consisted of a 10-minute 95° C. hotstart followed by 40 cycles of 25 seconds at 94° C., 45 seconds at 59° C., 45 seconds at 72° C., and completed with a 10-minute hold at 72° C. For each reaction, a mixture of 1 μL PCR product, 0.5 μL 400HD ROX size standard, and 12 μL Hi-Di Formamide was separated on an ABI 3100 Sequencer and analyzed in GeneScan 2.1.

Transwell Migration Assays. Activated PBLs or activated MILs (1.5×10⁵) were placed in the top well of a 4-μm 96-transwell plate (Millipore, Billerica, Mass.) and co-incubated at 37° C. with various concentrations of stromal cell-derived factor-1 (SDF-1; Peprotech, Rocky Hill, N.J.) in the bottom wells for 4 hours. Chemotaxis-mediated transmigration was determined by fluorescence-activated cell sorting analysis of CD3+ T cells in the bottom well.

Caspase-Cytoxicity Assay. This assay was done according to the protocol of the manufacturer (OncoImmunin Corp., Gaithersburg, Md.). In brief, autologous CD138⁺ plasma cells (2×106/mL) were stained with the phycoerythrin-labeled Target Marker in AIM-V for 1 hour at 37° C. in 5% CO₂, then washed thrice with AIM-V. During this hour of incubation, activated PBL and activated MIL effector cells were harvested following a 5-day stimulation with anti-CD3/CD28 antibody-conjugated magnetic beads and subsequent 2-day rest and resuspended in AIM-V. The target and effector cells were mixed at the desired effector to target ratios. The labeled targets and effectors were added together in 5 mL round-bottomed polystyrene tubes and centrifuged at 1,250 rpm for 5 minutes and then resuspended in either 75 μL of the FITC-labeled caspase substrate or in wash buffer (as a non-substrate control), and incubated for 3 hours at 37° C. in 5% CO₂. Following incubation, all samples were washed, resuspended in wash buffer, and analyzed using flow cytometry. Cleavage of the fluorogenic caspase substrate identified target CD138⁺ cells undergoing cytolysis.

Myeloma Progenitor Outgrowth Assay. Bone marrow mononuclear cells from myeloma patients were depleted of CD34⁺, CD138⁺, and CD3⁺ cells as previously reported (Matsui et al., Blood 2004; 103:2332-6.). The resulting negative fraction (5×10⁵ cells/mL) was then incubated with varying T-cell populations for 24 hours and then plated in methylcellulose-containing lymphocyte conditioned media. Colonies were scored at 2 weeks and confirmed as myeloma colonies by CD138+ staining and light chain restriction based on the patients' initial plasma cell population. Data shown for each group is the total number of outgrowth colonies.

### Example 2

### Preferential Expansion of Marrow-Infiltrating Lymphocytes Compared with Peripheral Blood Lymphocytes

In an effort to develop strategies to increase the efficacy of adoptive immunotherapy, we obtained and expanded MILs from multiple myeloma patients. MILs expanded to a greater extent than PBLs after a 5-day stimulation with the anti-CD3/CD28 antibody-conjugated magnetic beads. In the patients analyzed, the most pronounced difference was observed in CD4+ cells where the increase in the activated MILs exceeded the activated PBLs by more than 10-fold (activated MILs 27.6±8-fold expansion versus activated PBLs 2.8±1-fold expansion) whereas the absolute expansion of CD8+ cells was considerably less (activated MILs 15.3±7-fold, activated PBLs 3.6±2-fold). Enhanced proliferation of MILs over PBLs is consistent with a memory/effector phenotype. To test this, we examined the surface expression of CD40L and CD45RO at baseline and following activation. Interestingly, the baseline surface expression of both these markers was greater in MILs as compared with PBLs and the subsequent increase upon activation was more pronounced in the activated MILs. Furthermore, we also examined the impact of anti-CD3/CD28 expansion of MILs on the central memory phenotype (T_{CM}) which has been shown by others to be critical to the persistence of antigen specific T cell upon adoptive transfer, and showed that greater than 75% of the activated MILs possessed this phenotype. A major concern regarding the nonspecific stimulation of bone marrow from patients with multiple myeloma is the possibility of also expanding tumor cells. To address this, we stained for CD138⁺ plasma cells pre- and post-expansion and saw no increase in cell numbers after the 5-day stimulation. In fact, the final cultures showed undetectable levels of CD138⁺ cells confirming that the in vitro stimulation of lymphocytes cultures with anti-CD3/CD28 antibody-conjugated beads did not expand tumor cells. Taken together, these data confirm the enhanced proliferative capacity of MILs as compared with PBLs in the same patients when the cells are cultured using the conditions described in Materials and Methods.

### Example 3

### Activated Marrow-Infiltrating Lymphocytes Show a Skewed T-Cell Receptor Vβ Repertoire

To determine the effect of anti-CD3/CD28 bead stimulation on the T-cell repertoire of lymphocytes from the peripheral blood and marrow of patients with multiple myeloma, T-cell receptor Vβ spectratyping analysis was done on patients before and after bead activation. Unactivated PBLs and MILs both showed a similar degree of Vβ T-cell skewing with similar oligoclonal peaks observed in certain Vβ families. However, whereas activation with the anti-CD3/CD28 antibody-conjugated magnetic beads tended to normalize polyclonal Vβ T-cell repertoire in activated PBLs, oligoclonality persisted in the activated MILs. These results show the preservation of the skewed T-cell repertoire in MILs following activation and expansion as compared with activated PBLs, possibly suggestive of maintenance and/or enrichment of T cells with heightened tumor specificity.

### Example 4

### Activated Marrow-Infiltrating Lymphocytes Display Enhanced Antitumor Specificity Compared with Activated Peripheral Blood Lymphocytes

The propensity of MILs to retain a skewed T-cell repertoire profile might reflect the selective pressure maintained by the presence of tumor during bead activation. We thus sought to determine if functional differences existed between activated MILs and activated PBLs. Specifically, we evaluated the effect of in vitro activation with anti-CD3/CD28 antibody-conjugated magnetic beads on the tumor specificity of lymphocyte populations obtained from either compartment.

PBLs and MILs were fractionated using Ficoll Hypaque® Plus (Amersham). Briefly, peripheral blood was diluted 1:2 in HBSS and 30 ml or less of the dilution is layered onto 14 ml of Ficoll. Bone marrow was diluted 1:3 with HBSS and the same Ficoll layer was made. Cells were centrifuged at 1500 rpm for 30 minutes with the brake off at room temperature. expanded and activated by adding magnetic beads conjugated to anti-CD3/CD28 antibodies at a 3:1 ratio of beads to CD3⁺ cells. The beads were added directly to the fractionated bone marrow or peripheral blood mononuclear cells and removed after a 5-day stimulation. Following a 2-day rest, T-cell-mediated tumor specificity was assessed by examining the proliferative response to autologous CD138⁺ plasma cells. CD33⁺ myeloid cells or T cells alone served as controls. Proliferative responses of activated T cells were compared with unactivated T cells from both compartments (PBL and MIL). Whereas unactivated MILs showed minimal proliferation towards CD138⁺ cells, activated MILs showed significant tumor specificity with a 62-fold stimulation index, 121,692 (±11,916) cpm pulsed with CD138⁺ versus 1,918 (±75) cpm unpulsed activated MILs. In sharp contrast, activated PBLs showed only a 3.6-fold stimulation index of activated PBLs pulsed with CD138⁺ cells versus no antigen, 5,631 (±1,196) cpm versus 1,558 (±190) cpm), similar to the unactivated PBLs demonstrating a 3.4-fold stimulation index towards CD138⁺ cells versus no antigen. A major concern of directly activating MILs within the bone marrow microenvironment is the possibility of generating nonspecific T-cell responses to normal hematopoietic elements that would limit the applicability of this approach in a clinical setting. Examination of the impact of aMILs on the normal hematopoietic colony formation in the bone marrow was examined and no difference was observed in CFU-GM colony numbers in cultures grown in the presence or absence of aMILs. The absence of measurable T-cell reactivity towards autologous CD33⁺ myeloid cells over baseline shows the significant degree of tumor specificity that activated MILs possess. To determine if the antitumor effect was mediated via T cell receptor-major histocompatibility complex, engagement pan Class I and pan Class II antibodies were added to tumor specificity cultures. The addition of blocking Class I and Class II antibodies completely abrogated the proliferative response of activated MILs to autologous CD138+ cells.

### Example 5

### The Presence of Antigen During T-Cell Expansion May be Critical in Maintaining the Tumor Specificity of Activated Marrow-Infiltrating Lymphocytes

One major difference between the bone marrow and peripheral blood compartments of patients with multiple myeloma is the significantly greater tumor burden in the bone marrow compared with the peripheral blood. To evaluate the contribution of tumor within the bone marrow in enhancing tumor reactivity in the activated MILs, MILs were selected and added to the T-cell-depleted peripheral blood whereas peripheral blood T cells were selected and added to the T-cell-depleted bone marrow at greater than 90% purity. With this experimental design, MILs would be activated and expanded in the absence of any elements present within the marrow microenvironment. In contrast, if the marrow contained elements solely responsible for imparting and/or maintaining antigen specificity, we would expect to see augmentation of tumor specificity of the PBLs activated in the bone marrow environment. These groups were activated with anti-CD3/CD28 antibody-conjugated magnetic beads as previously described, rested for 2 days, and placed in a proliferation assay with autologous CD138⁺ cells, autologous myeloid CD33⁺ cells, or no antigen. The T-cell-depleted bone marrow reconstituted with PBL-derived T cells showed a similar degree of proliferation as the PBLs activated in the blood. In contrast, the group containing T-cell-depleted peripheral blood reconstituted with marrow-derived T cells (MILs) proliferated less than the activated MILs group.

We then sought to determine whether the reduced tumor specificity of bone marrow lymphocytes stimulated in the T-cell-depleted peripheral blood was due to a loss of critical marrow-derived elements or simply dependent on the absence of CD138+ cells. The anti-CD3/CD28 antibody-conjugated magnetic beads were added to the bone marrow cells and magnetically removed after an initial incubation period to isolate the bead-T cell complex. These enriched T cells were expanded for 5 days and rested for 2 days as before. MILs were analyzed for their ability to proliferate to autologous tumor (CD138⁺), autologous myeloid cells (CD33⁺), or no antigen. Maximal tumor-specific proliferation was achieved in the group expanded and activated in the presence of tumor, but tumor-specific proliferation was not completely lost in the MIL group lacking the tumor antigen. Taken together, these data show that the presence of antigen is likely critical during activation with the anti-CD3/CD28 antibody coated beads to maintain tumor specificity and highlight the importance of the bone marrow in myeloma in creating a permissive environment for the generation and maintenance of T cells with heightened tumor specificity.

### Example 6

### Activated Marrow-Infiltrating Lymphocytes Show Increased Stromal Cell-Derived Factor-1-Mediated Transwell Migration

Tumor-specific T cells can possess high affinities for their cognate antigen, but the ability to generate an effective immune response requires direct contact with the tumor in the proper context. One factor that may play a role in facilitating such interactions is SDF-1. It is a known bone marrow chemoattractant and its cognate receptor on T cells is CXCR-4. CXCR-4 expression was much greater on activated MILs than on activated PBLs. To examine whether differences in SDF-1-mediated responsiveness existed between MILs and PBLs, we did a transwell experiment. Activated MILs or activated PBLs were placed in the upper well of a transwell plate with SDF-1 in the bottom well at the indicated concentrations and harvested after 4 hours of incubation. The transmigration of activated MILs significantly exceeded that of activated PBLs when incubated with 100 ng/mL of SDF-1. No significant differences were seen using these conditions with the unactivated T cells. The significant trans-migratory ability of activated MILs and their pronounced up-regulation of CXCR-4 could play a critical role in facilitating trafficking of these activated T cells to marrow/tumor microenvironment that may be critical in establishing and/or maintaining tumor specificity.

### Example 7

### Activated Marrow-Infiltrating Lymphocytes Exhibit Greater Tumor-Specific Cytotoxicity as Compared with Activated Peripheral Blood Lymphocytes

To determine whether these cells effectively kill tumor, we sought to analyze their cytolytic capabilities. We used a cytotoxic fluorometric assay that measures the cleavage of a cell-permeable fluorogenic caspase substrate in cells undergoing apoptosis to analyze the differences in cytolytic capabilities of the activated MILs compared with the activated PBLs. This assay was chosen instead of the traditional cytotoxicity assay because of the inability to effectively label autologous plasma cells with chromium. In addition, the ability to specifically examine plasma cell cytotoxicity as opposed to generalized, nonspecific cell killing enables a more accurate determination of T-cell-mediated tumor cell killing. Furthermore, caspase cleavage represents an early event in cell death and thus serves as a more sensitive marker of cytotoxicity than the classic chromium release assay. Lymphocytes obtained from both compartments (peripheral blood and marrow) from patients were stimulated for 5 days with the anti-CD3/CD28 antibody-conjugated magnetic beads at a bead to T cell ratio of 3:1, and rested for 2 days. Autologous CD138⁺ cells (targets) were selected for each patient and fluorescently labeled with the Target Marker, then co-incubated with either activated MILs or activated PBLs (effectors) in the presence of a fluorogenic caspase substrate. Prior titration studies showed maximal cytotoxicity at an effector to target ratio of 5:1. This effector to target ratio was used in subsequent studies.

Following a 3-hour incubation the cells were washed and analyzed by flow cytometry. Cleavage of the caspase substrate increases the fluorescent intensity (in the FL-1 channel) of the dying CD138⁺ tumor cells. Activated MILs showed almost a 100-fold greater tumor-specific cytolytic function as compared with activated PBLs in an assay normalized for the same number of T cells (Patient 1: 22.3% activated MILs versus 0.26% activated PBLs; Patient 2: 51.5% versus 0.12%; and Patient 3: 42.3% versus 0.36%, respectively). The addition of pan-HLA-Class I and Class II antibodies completely blocked the cytotoxic ability of activated MILs. In the unactivated state, no cytotoxic activity was observed in PBLs and minimal activity in MILs. The absence of tumor-specific cytotoxicity of unstimulated lymphocytes shows the profound unresponsiveness present in this population and underscores the requirement for in vitro T-cell activation to generate tumoricidal activity.

### Example 8

### Activated Marrow-Infiltrating Lymphocytes Are Potent Inhibitors of Plasma Cell Outgrowth in a Clonogenic Assay

Multiple myeloma is a B-cell malignancy marked by an increase of terminally differentiated plasma cells with minimal self-renewing capabilities. We previously showed that clonogenic cells in multiple myeloma resemble post-germinal center B-cells rather than the terminally differentiated plasma cells (Matsui et al., 2004). For therapies to induce durable remissions they must significantly reduce both the terminally differentiated plasma cells as well as their self-renewing precursors. Due to activated MILs showing significant antitumor activity against plasma cells, we investigated whether they also inhibited the clonogenic outgrowth of myeloma precursors. Autologous bone marrow cells were co-incubated with PBLs, activated PBLs, MILs, or activated MILs, plated in methylcellulose, and evaluated for CD138⁺ light chain-restricted plasma cell colony formation. Activated MILs inhibited plasma cell colony formation by 86%, whereas PBLs, MILs, and activated PBLs had significantly less activity against clonogenic myeloma outgrowth (27%, 38%, and 47%, respectively). Similar inhibition of clonogenic outgrowth was also observed using the two myeloma cell lines, H929 and RPMI 8226, where activated MILs inhibited plasma cell colony outgrowth by 100% in the H929 cell line and by 84% in the RPMI 8226 line.

Considering the potent antitumor response on plasma cell clonogenic precursors, we sought to analyze whether this inhibitory response would also extend to normal hematopoietic precursors owing to such a finding precluding the ability to use this approach therapeutically. Normal hematopoietic granulocyte-macrophage colony-forming unit outgrowth was unaltered with the addition of activated PBLs or activated MILs, thus confirming the tumor specificity of activated MILs. Taken together, these data also show the greater antitumor activity of activated MILs over activated PBLs towards the plasma cell progenitors.

### Example 9

### Materials and Methods

Methods were performed using methods known to those of skill in the art and as provided in Serafini et al., 2006. J. Expt. Med. 203:2691-2702 which is incorporated herein by reference.

Cell lines. The following cell lines were used: CT26, a carcinogen-induced, undifferentiated colon carcinoma obtained from BALB/c mice (Griswold, D. P., and T. H. Corbett. 1975. A colon tumor model for anticancer agent evaluation. Cancer. 36:2441-2444); TS/A, a mouse mammary adenocarcinoma derived from BALB/c mice (Nanni, et al, 1983. Clin. Exp. Metastasis. 1:373-380); MCA₂O₃, a C57BL/6-derived fibrosarcoma (Spiess, et al., 1987. J. Natl. Cancer Inst. 79:1067-1075); and B16-GM, a C57BL/6 melanoma cell line genetically modified to secrete GM-CSF (Dranoff, et al, 1993. Proc. Natl. Acad. Sci. USA. 90:3539-3543). The 4T1-HA cell line was obtained by lentiviral transduction of 4T1 mammary carcinoma and was provided by K. Whartenby (Johns Hopkins University, Baltimore, Md.). These cell lines were grown in DMEM or RPMI 1640 (Invitrogen) with 10% FBS (Invitrogen). The C26GM cell line derived from the C26 colon carcinoma was genetically modified to produce GM-CSF (Bronte, et al., J. Immunol. 170:270-278) and was grown in the presence of 800 ng/ml G418.

Drugs and cytokines. Sildenafil (Pfizer) was dissolved in the drinking water (20 μg/kg/24 h), given i.p. daily where indicated (20 μg/kg/24 h), or added to the cell cultures (50 mg/ml). 2 μg/kg/24 h tadalafil (Lilly ICOS) was administered i.p. In vivo treatments started on day 0 unless otherwise indicated. N or NOHA and 1-NMMA (Calbiochem) were used at 500 μM in vitro.

Mice and in vivo experiments. 4-6-wk-old BALB/c and C57BL/6 mice were purchased from Harlan. C57BL/6-NOS2^{-/-} mice (strain B6; 129P2-Nos2tm1Lau) and control mice (strain B6; 129 PF2/J-100903) were purchased from the Jackson Laboratory. BALB/c-Rag-2^{-/-} (Shinkai, et al. 1992. Cell. 68:855-867), clone 4 mice transgenic for the H-2 Kd-restricted TCR recognizing the influenza virus, HA peptide (HAp512-520) TCR-transgenic (6.5) mice recognizing the HAp110-120 presented by I-Ed, and OT-II TCR-transgenic mice recognizing OVAp329-337 presented by I-Ab were all bred in the Johns Hopkins University animal facility. BALB/c-pIL-2/GFP mice were a gift of C. T. Weaver (University of Alabama, Tuscaloosa, Ala.) (Saparov, et al. 1999. Immunity. 11:271-280.). All mouse experiments were in accordance with protocols approved by the Animal Care and Use Committee of the Johns Hopkins University School of Medicine.

Tumor cells (0.5×10⁶) were injected s.c. in the inguinal area. Tumor measurements were performed with a caliper by measuring the largest diameter and its perpendicular length. The tumor size index is the average of the product of these diameters measured independently by two operators. Gr-1 depletion was performed by i.p. injection of 100 ng of anti-Gr-1 depleting antibody (clone RB6.8C5-18) per mouse on days 0, 3, and 6. CD8 depletion used 200 μg of the anti-CD8 depleting antibody (clone 2.43) on days 0, 2, 4, and 6. All of the experiments were performed at least twice with five mice per group unless otherwise indicated in the figures.

In vivo CTL assay. BALB/c splenocytes were stained either with 5 μM CFSE (Invitrogen) for 10 min at 37° C. and pulsed with the relevant AH-1 MHC class I peptide (corresponding to amino acids 423-431 of gp70, SPSYVYHQF; CFSE^{high} cells) or with 0.5 μM CFSE and pulsed with an irrelevant peptide (HA) as a control (CFSE^{low} cells). 10⁷ splenocytes (per population) were transferred i.v. to each host. The draining inguinal lymph nodes and spleens were harvested 40 h later and analyzed by FACS. Percent lysis was calculated as 1−CFSE^{high}/CFSE^{low} normalized by the same ratio in naive BALB/c mice (% lysis=100×{1−[(CFSE^{high}ₑₓₚ/CFSE^{low}ₑₓₚ)/(CFSE^{high}_{BALB/c}/CFSE^{low}_{BALB/c})]}).

Flow cytometry. Single cell suspensions from spleens or tumors were stained with PE-conjugated anti-mouse CD8 (CD8-PE; BD Biosciences), allophycocyanin-conjugated anti-mouse CD4 (CD4-allophycocyanin; BD Biosciences), FITC-conjugated anti-mouse CD11c, PE-conjugated anti-mouse B7.2, cychrome (Cy)-conjugated anti-mouse MHC class II, or with allophycocyanin-conjugated anti-mouse CD11b (BD Biosciences) and PE-conjugated anti-mouse Gr-1 (CD8-PE). IL-4Rα expression was determined on purified CD11b+ cells with PE-conjugated anti-mouse CD124 (BD Biosciences). Isotype-matched antibodies were used as controls, and live cells were gated based on 7-amino-actinomycin D, annexin V staining. Samples were run on a flow cytometer (FACSCalibur; BD Biosciences), and the data were analyzed using FCSexpress software (v 2.0; De Novo Software).

Cell purification. CD11b⁺ purification was performed with mouse CD 11b MicroBeads (Miltenyi Biotec). The positive and negative fractions were sorted with the LS columns according to the manufacturer's instructions. CD11c⁺ cells were positively selected from splenocytes of tumor-bearing mice using mouse CD11c MicroBeads (Miltenyi Biotec). CD4⁺ or CD8⁺ T cells were negatively selected, using the CD4⁺ or CD8⁺ T cell isolation kit (Miltenyi Biotec), from CD11b-depleted splenocytes. CD14 depletion of human PBMCs was obtained by adding PE-labeled, anti-human CD14 antibody (BD Biosciences). The CD14-negative population was collected using a cell sorter (FACS Vantage SE; BD Biosciences).

Suppressive assay. 2×10⁵ purified splenic or intratumoral CD11b⁺ cells were added to 10⁶ clonotypic CFSE-labeled splenocytes. They were then peptide pulsed and cultured for 3 d in 96-well flat-bottom plates. Sildenafil was added to the appropriate samples.

Proliferation assay. PBLs were obtained from MM or head and neck cancer patients after obtaining informed consent using an Institutional Review Board-approved protocol. Ficolled PBMCs were stimulated with anti-CD3/28 antibody-coated Dynal beads (3:1 bead/T cell ratio) for 5 d in a 96-well round-bottom plate and analyzed by flow cytometry. Sildenafil was added where indicated in the figures. Results are reported as fold change (number of activated cells/number of unactivated cells).

cGMP. cGMP was measured on purified CD11b+, CD11c+, CD4+, or CD8+ cells using the Cyclic GMP EIA kit (Cayman Chemical). Data analysis was performed with the workbooks available at http://www.caymanchem.com/neptune/servlet/neptune/template/analysis %2CEIA.vm/a/z. Data are expressed as mean±SE of quadruplicate wells.

Western blot. Lysates from purified cells were denaturated at 95° C. for 10 min and subjected to SDS-PAGE, and proteins were transferred overnight to polyvinylidene difluoride membranes. The membranes were incubated with a rabbit polyclonal anti-NOS2 antibody (Santa Cruz Biotechnology, Inc.), mouse anti-ARG1 antibody (a gift from Augusto C. Ochoa, Louisiana State University, New Orleans, La.), or a polyclonal rabbit anti-actin antibody (Sigma-Aldrich). Proteins were detected using the SuperSignal West Pico Chemiluminescent Substrate kit (Pierce Chemical Co.) according to the manufacturer's instructions

NO measurement. NO (nitric oxide) was measured using a nitrate/nitrite assay kit (Cayman Chemical) according to the manufacturer's instructions. Results were normalized to 106 cells. Data are from triplicate wells.

Arginase assay. The arginase assay was performed as previously described (8) on purified intratumoral MDSCs.

Statistical analysis. Bivariate Pearson and analysis of variance (ANOVA) analyses were performed using SPSS (v 7.0). All experiments were repeated at least twice, and all p-values were two sided (t test) or one sided (ANOVA).

### Example 10

### PDE-5 Inhibition Augments Immune-Mediated Antitumor Activity In Vivo

When administered in vitro, PDE-5 inhibition induces apoptosis in colon carcinoma and chronic lymphocytic leukemia cells. To determine whether similar effects could be observed in vivo, we used various transplantable mouse tumors, including CT26WT (a colon carcinoma), the more aggressive variant C26GM, TS/A (a mammary adenocarcinoma), and the MCA₂O₃ fibrosarcoma. PDE-5 inhibitors were administered starting on the day of tumor challenge. Sildenafil and tadalafil significantly delayed tumor outgrowth by 50 to 70% in immune-competent mice, although all mice ultimately died. Similar results were obtained even if sildenafil treatment was started on day 7 after tumor challenge in the CT26WT model. The fact that no difference in tumor outgrowth was seen between early versus late administration of sildenafil suggests that PDE-5 inhibition does not appreciably affect the early phases of tumor uptake but rather influences the later stages of tumor outgrowth. Because the addition of sildenafil to cultured CT26WT cells did not increase their apoptosis or affect their doubling time, we conclude that sildenafil does not have a direct antitumor effect but rather interferes in host-tumor interactions.

To confirm that the antitumor effect of PDE-5 inhibitors was immune mediated, the experiments were repeated in immune-compromised BALB/c-Rag-2^{-/-} mice. In these hosts, sildenafil demonstrated no antitumor efficacy. Because these mice lack T and B lymphocytes but have normal or enhanced NK and NKT activity, these results strongly suggest that the antitumor activity of PDE-5 inhibition in our models is primarily caused by an adaptive immune response with either minimal NK/NKT-mediated activity, direct tumor-induced apoptosis, or tumor angiogenesis inhibition. Evidence of an immune-mediated, antitumor effect of PDE-5 inhibition is further shown by tetramer analysis on splenocytes obtained from sildenafil-treated, CT26WT tumor-bearing mice, which revealed a higher number of CTLs specific for AH-1, a CT26 tumor-associated antigen, as compared with their untreated counterparts.

To confirm immune-mediated antitumor activity in the sildenafil-treated groups, we performed an in vivo cytotoxicity assay. BALB/c mice were injected with PBS (naive), vaccinated with γ-irradiated CT26WT or C26GM, or challenged with CT26WT or C26GM tumors on day 0. On day 12 (CT26WT) or day 5 (C26GM), all mice received carboxyfluorescein diacetate succinimidyl ester (CFSE)-labeled splenocytes pulsed with the MHC class I-restricted AH-1 peptide (CFSEhigh), admixed with CFSE-labeled splenocytes pulsed with the irrelevant hemagglutinin (HA)-peptide (CFSElow). In vivo T cell cytotoxicity was determined 40 h later. These time points were chosen based on the kinetics of tumor outgrowth observed, when tumor size significantly differed between untreated and sildenafil-treated mice. As expected, an endogenous AH1-specific immune response was observed in the vaccinated mice as compared with their tumor-bearing counterparts. PDE-5 inhibition in the vaccine-primed mice failed to augment antigen-specific CD8 responsiveness compared with no treatment. In contrast, tumor-bearing mice treated with sildenafil early after tumor challenge generated antigen-specific immunity that was significantly greater then that observed in their untreated counterparts and similar, or even superior, to that induced by vaccination.

Collectively, these data indicate that PDE-5 inhibitors can modulate antitumor immunity. Because the sildenafil-mediated antitumor immune response does not completely eradicate tumors, tumor escape mechanisms may be associated with their outgrowth. To test this hypothesis, the parental CT26WT cell line, as well as the CT26 tumor removed on day 24 from sildenafil-treated mice (either AH-1 pulsed or unpulsed), and BALB/c splenocytes were incubated with either AH-1 peptide-primed or tumor-primed effector T cells. Although effector T cells recognized the parental CT26WT line and released IFN-γ in the assay, they failed to recognize the sildenafil-derived tumor. Its recognition, however, was restored by loading the sildenafil-derived tumor with the AH-1 peptide. These results suggest that the immune response in sildenafil-treated mice does not result in complete tumor eradication but rather in the selection of antigen-escape variants.

### Example 11

### PDE-5 Inhibition Synergizes with Adoptive Cell Therapy (ACT) to Delay Tumor Outgrowth

Considering that sildenafil treatment significantly delayed tumor outgrowth but failed to eradicate it, we sought to determine whether combining sildenafil with tumor-specific CD8⁺ lymphocytes could enhance the therapeutic efficacy of ACT. On day 1 after tumor challenge, C26GM-bearing mice received purified CD8⁺ T cells derived from mice vaccinated with γ-irradiated C26GM cells. After adoptive transfer of these vaccine-primed CD8⁺ T cells, the mice were either treated with sildenafil or left untreated. Although adoptive transfer alone demonstrates no statistically meaningful antitumor effect compared with no treatment, PDE-5 inhibition significantly reduces tumor outgrowth. However, coupling adoptive immunotherapy with PDE-5 inhibition resulted in the greatest antitumor effect.

To extend this finding to other models, the experiment was repeated using the 4T1 mammary carcinoma genetically modified to express the influenza-derived HA as a model tumor antigen. Mice were challenged on day 0 with 0.5×10⁶ 4T1-HA cells. On the next day they received 3×10⁶ HA-specific naive CD8⁺ T cells. After adoptive transfer, the mice were either treated with sildenafil or left untreated. As with the C26GM experiments, no therapeutic benefit was observed with ACT alone, whereas sildenafil+ACT imparted the greatest antitumor benefit. Sildenafil treatment alone, instead, substantially delayed tumor progression in immunocompetent BALB/c mice but not in immune-deficient Rag-2^{-/-} mice. These data confirm the immunomodulatory properties of PDE-5 inhibitors in augmenting the therapeutic efficacy of ACT and demonstrate a role of sildenafil in modifying the tumor microenvironment rendering it more susceptible to CTL-mediated cytotoxicity.

### Example 12

### PDE-5 Inhibition Increases Tumor-Infiltrating CD8+ T Cells

To exert a measurable antitumor effect, tumor-specific T cells must be present in sufficient numbers and capable of trafficking to their targets. A direct correlation exists between the number of tumor-infiltrating lymphocytes (TILs) and a favorable clinical outcome, as demonstrated in patients with metastatic ovarian cancer. Furthermore, the functional status of TILs has been correlated with a favorable prognosis in various human malignancies. Because PDE-5 inhibition augments antitumor immunity, we asked whether sildenafil treatment altered both the number and activation state of TILs. Histological examination of CT26WT tumors revealed a greater intratumoral cellular infiltrate in the sildenafil-treated mice compared with the untreated controls. To better evaluate these differences, C26GM-bearing mice received either tumor-primed or no T cells followed by sildenafil treatment or no additional therapy. The tumors were excised 9 days later, and single cell suspensions were obtained. The T cell infiltrate was analyzed by flow cytometry for CD4⁺ and CD8⁺ T cells. This approach enabled us to accurately examine the entire tumor mass and reliably quantify the infiltrating lymphocytic population. Although no increase in CD4⁺ T cells was observed with PDE-5 inhibition, sildenafil treatment greatly increased CD8⁺ intratumoral infiltration with up-regulation of the activation markers CD69 and CD25. There were no differences in activation markers between the sildenafil-treated group and sildenafil+ACT, whereas a significant increase in intratumoral T cells were observed in the sildenafil+ACT-treated group compared with sildenafil alone.

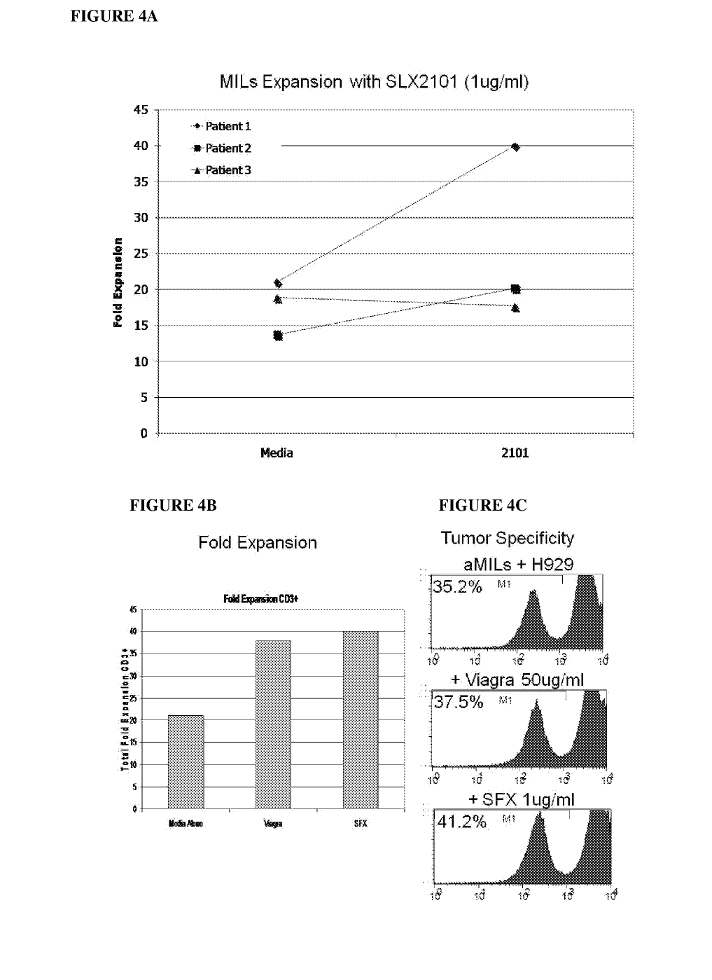

To determine whether the immunomodulatory effect of PDE-5 inhibition affected T cell activation within the tumor microenvironment, we examined IL-2 production by TILs using a transgenic mouse in which expression of GFP is under an IL-2 promoter (BALB/c-IL-2p/GFP) (Saparov, et al., 1999). In this model, T cell stimulation activates the IL-2 promoter and results in expression of the reporter transgene, GFP, which is easily detectable by flow cytometry. C26GM-primed BALB/c-IL-2p/GFP splenocytes were adoptively transferred into tumor-bearing recipients that were either left untreated or treated with sildenafil for 9 days. Single cell suspensions of the tumor-infiltrating CD8⁺ T cells were analyzed by flow cytometry for GFP expression. Adoptively transferred, vaccine-primed T cells were activated in the tumor microenvironment only in the presence of PDE-5 inhibition, whereas in its absence they produced no IL-2 and, hence, were bona fide anergic T cells. To further prove that these effects were dependent on CD8+ T cells, mice were challenged with C26GM and were (a) left untreated, (b) given sildenafil, (c) given an anti-CD8+ depleting antibody, or (d) given both sildenafil and the CD8+ depleting antibody. Sildenafil treatment again demonstrated a statistically significant reduction in tumor outgrowth, an effect completely abrogated by CD8+ depletion (FIG. 4 E). These experiments demonstrate that PDE-5 inhibition enhances the tumor-specific T cell response, increases intratumoral T cell infiltration and activation, and underscores the role of CD8+ T cells in sildenafil-mediated antitumor responses.

### Example 13

### In Vitro Sildenafil Down-Regulates the Myeloid Suppressor Cell Suppressive Marker IL-4Rα

The experiments described thus far demonstrate the ability of PDE-5 inhibition to prime/augment antitumor immunity. Yet the mechanisms resulting in T cell activation are unknown. Because PDE-5 is expressed in various cells of the immune system (including DCs, macrophages, and T cells), PDE-5 inhibitors could putatively target these various populations. Furthermore, numerous factors are present in tumor-bearing hosts that could impair the generation of an effective immune response such as the defective maturation of DCs, the accumulation of suppressive MDSCs, T cell anergy, and/or the accumulation of T reg cells. These populations were, therefore, examined separately. Flow cytometric analysis of sildenafil-treated or untreated mice failed to reveal important differences in DC maturation, CD4⁺Foxp3⁺ T reg cells, or CD11b⁺/Gr-1⁺ MDSC accumulation. We evaluated the effect of in vitro PDE-5 inhibition on purified CD11c⁺, CD11b⁺, CD4⁺, and CD8⁺ cells isolated from C26GM tumor-bearing mice. This enabled us to examine purified populations, thereby eliminating the potential for exogenous influences. Although sildenafil treatment increased the intracellular concentration of cGMP in all the populations examined, it had no effect on the proliferation of purified CD4+ and CD8+ T cells stimulated with either ConA or anti-CD3/CD28 antibody-conjugated magnetic beads, nor on CD11c maturation as determined by B7.2 or MHC class II up-regulation. It did, however, demonstrate the ability to reverse the suppressive phenotype of MDSCs. In light of recent data identifying IL-4Rα as a functional suppressive marker for MDSCs, we examined IL-4Rα expression on CD11b⁺ cells cultured in the presence or absence of sildenafil. Sildenafil was shown to significantly decrease IL-4Rα expression on MDSCs, suggesting a down-regulation of their suppressive pathways

### Example 14

### In Vivo PDE-5 Inhibition Down-Regulates Tumor-Associated MDSC Suppressive Pathways

Although the hallmark feature of MDSCs is immunosuppression, emerging data reveal that the degree of immunosuppression varies among populations of MDSCs isolated from different organs, with intratumoral MDSCs being the most immunosuppressive. Interestingly, these MDSCs express greater levels of NOS2 and ARG1 than their splenic counterparts. ARG1 expression is mainly regulated by the STAT-6-IL-4Rα pathway. We recently correlated IL-4Rα expression on CD11b⁺/Gr-1⁺ with an immunosuppressive phenotype, and our in vitro data indicate that sildenafil down-regulates IL-4Rα on MDSCs. We then asked whether in vivo PDE-5 inhibition reduced ARG1 and NOS2 and down-regulated IL-4Rα in tumor-associated MDSCs. BALB/c mice were challenged with CT26WT, and half were treated with sildenafil. Mice were killed 15 d later, and intratumoral MDSCs were obtained. Sildenafil increased cGMP, reduced IL-4Rα expression, and down-regulated NOS2 and ARG1 expression and reduced their enzymatic activity in the intratumoral MDSCs. Considering that ARG1 and NOS2 are key enzymes in MDSC suppressive pathways, these findings support the hypothesis that PDE-5 inhibition is a novel pharmacologic approach to regulate MDSC-mediated immunosuppressive pathways.

Gr-1⁺ cells are known to facilitate tumor outgrowth. As such, it is conceivable that strategies seeking to eliminate this population may have a measurable antitumor effect. In certain tumor models, the Gr-1 depleting antibody inhibited tumor outgrowth even in the absence of T cells, although the antitumor effect was more pronounced in immune competent mice. In light of these results and to verify that MDSCs are the target of sildenafil-mediated antitumor activity, we examined the effect of antibody-mediated MDSC depletion in combination with PDE-5 inhibition in vivo. BALB/c mice were challenged on day 0 with C26GM tumor and were (a) left untreated; (b) injected with 100 ng of anti-Gr-1 antibody i.p. on days 0, 3, and 6; (c) treated with sildenafil; or (d) treated with a combination of the two treatments. Gr-1 depletion delayed tumor outgrowth similarly to PDE-5 inhibition, whereas no synergistic effect was seen with the combination. Collectively, these data demonstrate the immunosuppressive nature of Gr-1⁺ cells and the ability of PDE-5 inhibition to reduce their suppressive phenotype in vivo.

### Example 15

### PDE-5 Inhibition Abrogates MDSC Suppressive Activity

Freshly isolated MDSCs suppress the in vitro proliferation of activated lymphocytes.

Interestingly, the suppressive mechanisms appear to be strain specific. In the Th1 cell prone strain C57BL/6, it is mediated by NOS2 through NO production, whereas, in the mixed Th1/Th2 cell BALB/c strain, suppression requires peroxynitrite formation via ARG1 and NOS2 coexpression or 1-arginine depletion secondary to ARG1 overexpression. Reductions of both ARG1 and NOS2 expression via PDE-5 inhibition should affect both suppressive pathways, resulting in less MDSC-mediated immunosuppression and, therefore, enhanced antigen-specific T cell proliferation. To test this hypothesis, tumor-derived CD11b⁺MDSCs were isolated from C26GM-bearing BALB/c mice. MDSC suppressive activity was determined by admixing MDSCs with CFSE-labeled HA-specific CD8⁺ (clone 4) or CD4+ (6.5) T cells pulsed with their relevant peptide in the presence or absence of sildenafil. Although the addition of tumor-derived MDSCs significantly impaired antigen-specific T cell proliferation as demonstrated by the low percentage of CFSE^{low} clonotypic T cells, sildenafil almost completely restored both CD4⁺ and CD8⁺ responsiveness of these antigen-specific T cells.

The absence of sildenafil-mediated enhancement in T cell function in the groups lacking CD11b+ cells underscores the targeted role of sildenafil on the MDSC population. Because in a Th1 cell-prone environment MDSC suppression is only NOS2 dependent, we examined the role of PDE-5 in MDSCs in a C57BL/6 background where NOS2^{-/-} mice are also available. CD11b+MDSCs were isolated from either C57BL/6-NOS2″ or B16GM-bearing C57BL/6-NOS2^{-/-} B16GM melanoma-bearing mice. A suppression assay was performed by stimulating OVA-specific CD4⁺ T cells with the relevant peptide in the presence or absence of MDSCs obtained from either NOS2^{+/+} or NOS2^{-/-} tumor-bearing mice (FIG. 7 C). Although the addition of C57BL/6-NOS2^{+/+} MDSCs induced considerable T cell suppression, no suppression was observed with MDSCs from NOS2^{-/-} mice. Furthermore, although PDE-5 inhibition reversed MDSC suppression in NOS2^{-/-} mice, sildenafil failed to augment T cell responsiveness in the NOS2^{-/-}-derived MDSC suppression assay. These results confirm the role of NOS2 in MDSC-mediated T cell suppression and underscore the ability of PDE-5 inhibition to reverse the two major suppressive pathways in MDSCs (ARG1 and NOS2).

### Example 16

### PDE-5 Inhibition Restores T Cell Proliferation in MM and Head and Neck Cancer Patients

Having demonstrated that PDE-5 inhibition can impair MDSC suppressive mechanisms in BALB/c and C57BL/6 tumor-bearing mice, we next sought to determine whether similar results could be obtained in humans. Head and neck cancers are known to be highly immunosuppressive. Their high levels of GM-CSF production are likely the major mediator of immune suppression observed in these patients and are probably responsible for the intratumoral infiltration by MDSCs. In fact, peripheral blood lymphocytes (PBLs) from these patients are functionally impaired in their ability to be activated and to proliferate upon stimulation. Similar results were also seen in prostate cancer and in nonsmall cell cancer. Although this anergic state in solid tumors may be attributable to the ARG1- and/or NOS-dependent suppressive activity of MDSCs, MDSC-mediated immunosuppression has not been previously reported in hematological malignancies. PBMCs from MM patients were stimulated with anti-CD3/CD28 antibody-coated beads in the presence of N-(omega)-hydroxy-nor-1-arginine (N or NOHA; an ARG1-specific inhibitor), NG-monomethyl-1-arginine (1-NMMA; an NOS2 inhibitor), both inhibitors, or neither.

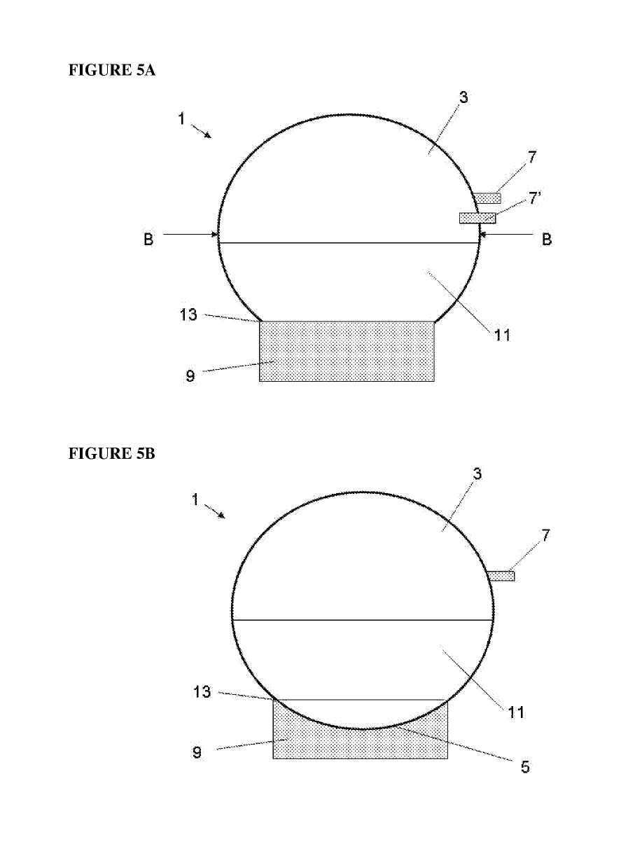

T cell expansion was considerably enhanced in the presence of both N or NOHA and 1-NMMA, whereas the single inhibitors failed to increase T cell proliferation over the baseline. Interestingly, sildenafil yielded results equivalent to the combination of N or NOHA and 1-NMMA. These results suggest involvement of both ARG1 and NOS2 in MDSC-mediated immunosuppression in myeloma and confirm the in vitro results demonstrating the ability of PDE-5 inhibitors to affect both pathways. Recent data from our lab identified human MDSCs as ARG⁺, CD14⁺ cells. We therefore examined T cell expansion in CD14⁺-depleted PBMCs under the same conditions. Although CD14⁺ depletion alone increased CD3⁺ T cell expansion fourfold, pharmacologic inhibitors failed to further enhance proliferation, suggesting that CD14⁺ cells are the mediators of ARG1- and NOS2-mediated immunosuppression in MM. As seen with purified mouse T cells (FIG. 5 B), sildenafil also failed to enhance the CD3⁺ T cell proliferation of CD14-depleted PBMCs from cancer patients.

We next sought to determine whether we could restore T cell proliferation of PBMCs isolated from head and neck and MM patients stimulated with anti-CD3/CD28 antibody-coated beads in the presence or absence of sildenafil. The expansion of CD4⁺ and CD8⁺ T cells obtained from MM or head and neck patients was significantly less than that observed using healthy donors. In contrast, in the presence of sildenafil, PBMCs from MM and head and neck patients expanded similarly to sildenafil-treated PBMCs from healthy donors. Interestingly, qualitative differences in T cell expansion emerged between the two malignancies. In head and neck patients, CD8+ T cell proliferation is restored with PDE-5 inhibition while CD4⁺ lymphocyte expansion in the same condition is only partially augmented. In contrast, CD4⁺ T cell proliferation is restored and minimal effects are seen on CD8+ lymphocyte expansion with PDE-5 blockade in myeloma. In summary, these data confirm that PDE-5 inhibition can augment immune responsiveness through its effect on an accessory CD14+ population. Moreover, they suggest that the same immunosuppressive mechanisms found in mice are conserved in human malignancies and that PDE-5 can be a useful therapeutic approach to enhance tumor-specific immunotherapy.

### Example 17

### Use of the NOD/SCID Mouse Model for Analysis of In Vivo T Cell Trafficking and Anti-Tumor Efficacy

The scope of the clinical trial related to the experiments provided herein is to determine whether we can effectively activate and expand myeloma specific MILs, safely infuse them in the setting of a standard high dose autologous peripheral stem cell transplant and determine anti-tumor immunity. Preliminary studies were performed in the NOD/SCID mouse model to mimic this disease and therapeutic intervention.

To model the in vivo T cell trafficking and anti-tumor efficacy, we utilized the immune compromised NOD/SCID mice which were challenged with the human myeloma cell line, H929. This cell line was chosen because of the variety of tumor associated antigens expressed including MAGE-A1, MAGE-A2, LAGE-1, NY-ESO-1, FGFR-3. In addition, it is HLA-A2⁺ us to utilize HLA-A2⁺ restricted T-cell that would minimize the likely of non-specific allo-reactivity. The studies we performed compared the tumor specificity properties and trafficking of activated and expanded MILs to peripheral T-cells as well as document the safety of administration of these cells in the setting of multiple myeloma.

Two animal studies were performed to study the safety and efficacy of the infusion of activated marrow infiltrating lymphocytes (aMILs) in vivo. In the first study, three doses of T-cells were administered on day 18 following intravenous tumor challenge but before measurable tumor could be detected in the serum.

### Experiment 1

Day 0/200XRT [symbol] Day 1/5×10⁶ H929iv→Day 17/200XRT [symbol] Day 18/^{±} aMIL
In the second study T cells were administered 30 days following tumor challenge at a time in which detectable tumor was present.

### Experiment 2

Day 0/200XRT [symbol] Day 1/5×10⁶ H929iv→Day 30/200XRT [symbol] Day 31/^{±} aMIL
In both experiments, the mice underwent total body irradiation (TBI) with 200 cGy 12 hours to kill bone marrow prior to i.v. injection of 5×10⁶ H929 cells to mimic multiple myeloma. A second dose of radiation was given on day 17 or 30 prior to the adoptive transfer of aMIL to create space for the T cells to properly engraft.

In the first experiment, the mice received an additional 200 cGy 12 hours prior to injection with 200 ul HBSS (the carrier solution), activated peripheral blood lymphocytes (aPBLs) T-cells, or activated marrow infiltrating lymphocytes (aMILs) at one of three dosages, 1×10⁶, 2.5×10⁶, or 5×10⁶.

In the second experiment, T-cell transfer occurred after a measurable tumor was established (as determined by the human Kappa Light Chain ELISA analysis using a commercially available kit (R&D Systems, Minneapolis, Minn.). The T cell dose in all was 1×10⁶ cells. In the second experiment we also evaluated the effect of the infusion of aMILs or aPBLs in a tumor-free setting to confirm the ability of T cell trafficking to the marrow. Tumor burden was assessed in real-time through determination of circulating human kappa light chain (produced and secreted by the human H929 myeloma cell lines). The endpoint of these experiments was survival. Upon their death the mice were dissected and bone marrow and spleen were evaluated for the evidence of tumor and infiltrating human T-cells as well as visually evaluated for infection and inflammation.

### Results—Experiment 1

These experiments attempted to address several issues. We had previously shown that MILs express the chemokine receptor CXCR4 in much higher levels than PBLs. SDF-1 is the cognate receptor of CXCR4 and is expressed at high levels by the bone marrow stroma. Therefore, we hypothesized that CXCR4 may be critical in T cell trafficking to the bone marrow and as such MILs would more effectively home to the marrow than would PBLs. Second, activated MILs (aMILs) showed a considerable tumor specific cytotoxicity compared to aPBLs. As such, we should see a measurable anti-tumor benefit of aMILs compared to aPBLs. Finally, to determine the optimal clinically effective T cell dose, we tested three doses: 5×10⁶/mouse, 2.5×10⁶/mouse and 1×10⁶/mouse. These doses, which equate to 1.7×10⁸/kg, 8.3×10⁷/kg, and 3.3×10⁷/kg in humans could be easily achieved clinically.

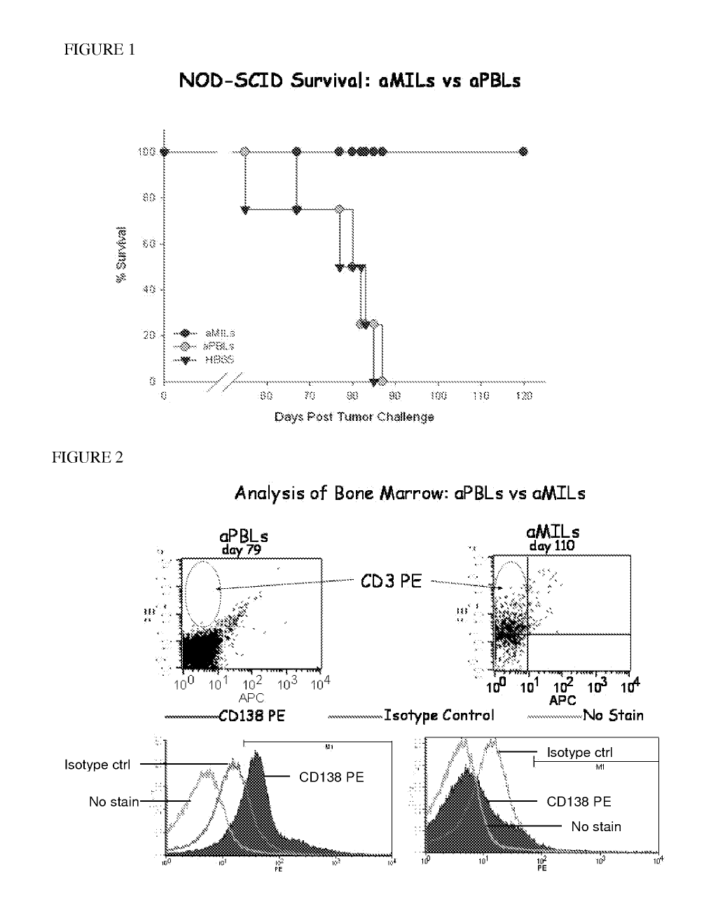

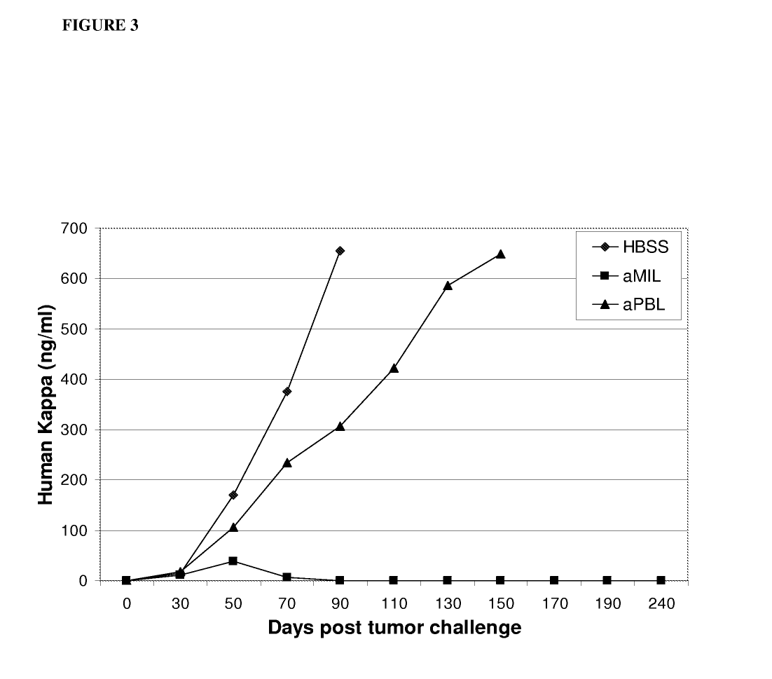

No survival difference was seen between mice that received aPBLs compared to the control group that received HBSS with all mice eventually dying of myeloma. In contrast, no deaths were observed in the mice challenged with aMILs during the course of the experiment (FIG. 1). The mice that received aMILs were euthanized on Day 120 whereas the other mice were analyzed prior to euthanization at the moment of significant tumor burden.

For all mice, the bone marrow and spleens were harvested and studied for evidence of T-cell and tumor infiltration. No T cells were seen in the bone marrow of the aPBL mice. Whereas, mice that received aMILs showed a significant number of human CD3 cells in their bone marrow (FIG. 2). Conversely, mice that received aPBLs showed a considerable amount of tumor, as stained by human CD138, while mice that received aMILs showed no evidence of tumor (FIG. 2). The spleens from all groups showed no evidence of either T-cell or tumor infiltration.

In experiment 1 we observed aMILs, but not aPBLs, in the bone marrow of the mice up to 100 days after i.v. injection. These data confirmed earlier data showing that aMILs express high levels of CXCR4, the cognate receptor of SDF-1 which is expressed in high levels in the stroma of the bone marrow, and therefore could effectively enable T cell homing to the marrow micro-environment. Interestingly, no tumor was found in the bone marrow of mice receiving aMILs, while aPBL mice had a large tumor burden in the marrow.

Another aspect of these studies was to determine the safety of adoptive T cell transfer of aMILs. There was no evidence of infection, inflammation or any other negative effect either at the injection site or throughout the mice upon dissection. The entire mouse was dissected and inspected for infection. All mice that died had extensive tumor burden and those that failed to show signs of tumor progression were euthanized after 120 days.

### Results—Experiment 2

In the first in vivo experiment we showed that aMILs could prevent the outgrowth of tumor in a mouse that did not show a quantitatively established tumor burden. In the second experiment we delayed adoptive T cell until there was evidence of measurable disease. Before activated MILs were administered, we demonstrated by ELISA for the human κ light chain that the tumor was in fact established at the time of the activated MIL administration. Furthermore, we also had groups of activated MIL therapy, only without tumor, to confirm the ability of aMILs to traffic independently of tumor burden.

Activated MILs again imparted significant anti-tumor efficacy even with this significantly higher tumor burden. Of note, the deaths reported in the aMILs group was due to an error in the animal facility in removing antibiotic water from the mice for several days resulting in infectious-related mortality and not a result of tumor progression. At 220 days, the aMIL treated mice still show no evidence of tumor progression, confirming the significant anti-tumor efficacy of adoptive immunotherapy with this approach.

Mature plasma cells secrete a kappa Light chain protein that can be measured with ease using a commercially available ELISA kit (R&D Systems). NOD/SCID mice were irradiated with 200 rads and then 1×10⁶ tumor cells were introduced i.v. Prior to injection and every five days thereafter the mice were tail-bled and plasma was isolated. Plasma was assayed for the presence of human kappa light chain ELISA kit for detection of measurable protein. Detection of human kappa light chain is indicative of the presence of tumor in the mice. In this experiment the kappa light chain was detected on D30 of the experiment. Mice then received another 200 rads to make space for T cells and were either injected with HBSS (saline as a negative control), aPBLs (1×10⁶ cells/mouse) or aMILs (1×10⁶ cells/mouse). Each five days thereafter the mice were tail-bled to determine the amount of human kappa light chain protein. Increased amounts of the protein indicate increased tumor outgrowth. The mice that received HBSS had continued tumor outgrowth and expired when the amount of protein detected was approximately 650 ng/ml. Mice that received aPBLs showed a short lag in tumor outgrowth, but by day 150 all mice had reached the roughly 650 ng/ml and succumbed to the tumor. In sharp contrast mice that were injected with aMILs saw a small increase in kappa light chain production that was undetectable by day 70, after which day no tumor was detectable. These mice survived over 250 days and were euthanized only for experimental reasons. These mice had detectable human CD3 cells but no tumor (CD38⁺/CD138⁺) in the bone marrow (data not shown).

In conclusion, the two NOD/SCID animal experiments support our in vitro data by demonstrating the significant anti-tumor efficacy of aMILs in the treatment of multiple myeloma. Furthermore, they failed to show any significant adverse reactions. Taken together, these data provide the rationale for the clinical design of the proposed protocol and appear to be void of any measurable toxicity.

### Example 18

### Optimization of Culture Conditions for Large Scale Expansion of MILs

Culturing of aMILs under conditions to allow for expansion and activation to provide sufficient cell numbers to allow for treatment of humans was found to require very specific culture conditions particularly in regard to the shape of the container in which the initial culturing was performed.

In order to culture cells for therapeutic use, closed systems (e.g., bags) must be used to both prevent contamination of the cells and contamination of the culture area. Prior to development of the culture method and device provided herein, many methods for activation and expansion of MILs were tested and found to be unsuccessful. In all experiments, cells were cultured in XVIVO 15 supplemented with 2% human AB serum and 1% Glutamax. Anti-CD3/CD28 antibody coated beads were added at a ratio of 3:1, beads: cells. The volume of the culture was 40 ml at a concentration of 2×10⁶ cells/ml in a culture bag for use in culturing up to 500 ml.

First, cells were added to a flat culture bag at three different concentrations, 0.5×10⁶ cells, 2×10⁶ cells, 4×10⁶ cells and tested for expansion as compared to control cells in small scale culture. In each of the large scale cultures, 0-10 fold MIL expansion was observed as compared to 50-100 fold MIL expansion in the small control culture.

Modification of bag shape and orientation were tested to identify large scale culture conditions. First, 2×10⁶ cells were placed in a bag and the flat bag was held between two clamps placed on the bag to form the bag into a shape approximating a flattened cylinder. No improvement in expansion was observed as compared to growing the cells flat in a bag. The same number of cells were placed in a bag that was hung vertically. Again, no improvement of expansion was observed over previous methods. In each case, the corresponding small control culture, expected levels of MIL expansion were observed.

Expansion in large scale culture improved upon fastening the ends of the culture bag towards the center of the bag to provide a wide U-like shape. The cells in the large scale culture were found to expand 0-25 fold as compared to the small culture where 50-80 fold expansion was observed.

Having determined that the rounded shape promoted expansion of the MILs in large scale culture, after placing the cells and media in the bag, the bag was inflated with ambient air until the bag was rigid to provide a rounded surface. Expansion was again improved, to 10-40 fold, which was still substantially less than that observed in the small culture which demonstrated 50-80 fold expansion.

Finally, cells (2×10⁶) were placed in a bag with media and filled with air as under the prior conditions and placed on a circular support. Expansion comparable to that observed in the small control culture was finally observed in the small culture.

### Example 19

### Ex Vivo aMILs Expansion with PDE-5 Inhibitor and Tumor Specificity

Cancer bearing hosts are plagued by a series of immunosuppressive mechanisms that together result in significant impairment of immune responsiveness. One mechanism gaining significant attention is induced by myeloid derived suppressor cells (MDSCs). These cells are induced by the tumor microenvironment, possess an immature monocyte/granulocyte phenotype, and impart their immune suppression through upregulation of arginase-1 (Arg1) and nitric oxide synthase-2 (NOS2). We have recently shown that phosphodiesterase-5 (PDE-5) inhibitors can reverse the MDSC-mediated immune suppression. Specifically, they act by down-modulating both Arg1 and NOS2 activity and not by eliminating this population of cells. In murine models, we have demonstrated that PDE-5 blockade can significantly restore T cell function, reduce the induction of regulatory T cells (Tregs) and augment immune mediated anti-tumor immunity.

Our studies have revealed the presence of MDSCs in myeloma patients both untreated or with advanced disease. As such, we examined whether PDE-5 blockade could augment our ability to expand MILs. As shown in FIG. 4, MILs from 3 patients were expanded with anti-CD3/CD28 antibody-conjugated magnetic beads either in the absence or presence of the PDE-5-inhibitor SLX 2101. Increases in T cell expansion ranged from 0 to 90% increase. We have previously shown that in the absence of MDSCs, the addition of a PDE-5 inhibitor does not augment T cell responsiveness. This is likely the case with Patient 3.

In a separate experiment, expansion of MILs was compared in cultures without PDE-inhibitor, with sildenafil at 50 μg/ml (noted as Viagra in the figure), and with SLX2101 at 1 μg/ml (noted as SFX in the figure). An expansion about 2-fold greater was observed when SLX-2101 is added (FIG. 4B).

A tumor specific assay was done with these cells, admixing the irradiated and HLA-matched myeloma cell line, H929, to the expanded MILs. The MILs were labeled with carboxyfluorescein diacetate succinimidyl ester (CFSE) according to manufacturer's protocol. CFSE consists of a fluorescent molecule containing a succinimydyl ester functional group and two acetate moieties. CFSE diffuses freely inside the cells and intracellular esterases cleave the acetate groups converting it to a fluorescent, membrane impermeable dye. This dye is not transferred to adjacent cells. CFSE is retained by the cell in the cytoplasm and does not adversely affect cellular function. During each round of cell division, relative fluorescence intensity of the dye is decreased by half and cellular division can be analyzed by the decrease of intensity of the cells in the FL-1 channel of a flow cytometer.

Cells that are less CFSE bright have divided in the presence of tumor and are therefore more tumor specific. A higher tumor specificity was observed in the cells treated with the PDE-5 inhibitor as compared to not treated with the PDE-5 inhibitor (FIG. 4C). There is a slight increase in overall tumor specificity with the addition of the SLX-2101 (from 35.2% CFSE^{low} in media alone—41.2% CFSE^{low} T-cells in the SLX-2101 group).

We have also shown that the addition of the inhibitor in such a mixed MDSC patient population does not necessarily impair T cell expansion. Considering that most of the patients we have examined thus far show evidence of MDSCs, the addition of a PDE-5 inhibitor, such as SLX-2101 significantly increases expansion MILs in most subjects, while not likely causing any harm in the population as a whole, and potentially increases our overall cell yield.

### Example 21

### Large Scale Processing, Separation, and Cryopreservation of aMILs

Bone marrow was aspirated into multiple, labeled syringes containing heparin. After aspiration, the syringes were stored overnight at room temperature. After storage, the contents of the syringes were pooled into a sterile vessel and samples removed for initial quality control testing. The bone marrow was enriched for mononuclear cells (MNCs) using lymphocyte separation media (LSM) and centrifugation utilizing a COBE Spectra. All of the cells in the gradient were collected down to the red blood cells and washed using HBSS. These mononuclear cells (MNCs) were then cryopreserved in bags (clinical product) and vial(s) (for subsequent quality control (QC) studies) using a hetastarch-based cryoprotectant supplemented with 2% HSA and 5% DMSO. The QC vial was thawed to determine the CD3⁺ cell and CD38⁺/138⁺ cell content of the MNC product. The specific method of bone marrow collection is not a limitation of the invention as long as the method provides viable, selected bone marrow cells for activation and expansion that contains CD3⁺ cells, tumor cells and other MNCs present in the bone marrow microenvironment.

The activation procedure was based on the requirements for both CD3 and CD28 receptor engagement in the activation of T cells. In the manufacturing process, T cells were stimulated ex vivo using monoclonal antibodies bound to magnetic beads that bind to the CD3 and CD28 molecules expressed on the surface of T cells. The antibodies are attached to paramagnetic particles, ClinExVivo™ CD3/CD28 Paramagnetic Particles, thereby creating artificial Antigen Presenting Cells.

To reverse the suppressive effect of MDSCs in the culture, SLx-2101 (which has been used in other Phase I and Phase Ha clinical trials) was added to the media on Day 0. The final concentration of the dissolved, sterile filtered drug was 1 ug/ml. To prepare the drug for inclusion in the expansion media, 5 mg tablets of SLx-2101 were dissolved in aMILs Base Medium (see below for media formulation details) and sterile filtered immediately prior to use. Alternatively, the drug can be obtained in powder form and added to the culture.

On Days 0 to 3 of production, aMILs Base Medium was used. aMILs Base Medium was prepared by adding the following reagents to a 1 L bottle of OpTmizer™ T-Cell Expansion Medium prior to filtration using a 0.22 micron filter:

26 mL of OpTmizer™ supplement

10 mL of GlutaMAX™

20 mL of Human AB serum

The cryopreserved MNC product was thawed in a 37±1° C. water bath. The thawed cells were slowly diluted with aMILs Base Medium, transferred to Lifecell® culture bag(s) further diluted with Base Medium containing 1 ug/ml of SLx-2101. QC samples were also thawed to determine the number of CD3⁺ T cells. T cells were incubated with CD3/CD28 paramagnetic particles at a bead to CD3⁺ cell ratio of 3 to 1 for one hour at room temperature on a LabNet rocker. The culture bag(s) were then placed on the support (donut device) and incubated at 37±1° C., 5±1% CO₂ and >85% humidity in a static culture. On Day 3 of expansion, an equal volume of aMILs Base Medium was be added to the cells.

On Day 4, aMILs Base Medium was added to either double the total volume of the culture or cap the culture volume at 2 L per 3 L culture bag whichever is the lower volume. Pluronic F68 was added to the media so that the final concentration of Pluronic F68 was 0.02%. The culture bag was incubated for another day at 37±1° C., 5±1% CO₂ and >85% humidity.

On Day 5, a sample was obtained for a cell count and sterility. On this day the cell suspension was transferred to the WAVE Bioreactor™ Disposable Cell Culture System and IL-2 was added to the expansion at a final concentration of 300 IU/ml. The volume in the WAVE bag was at a minimum 1.2 L and at a maximum 4.0 L. Additionally, the system was programmed so that 520 gm of aMILs Base Medium supplemented with 0.02% Pluronic F68 and IL-2 (final concentration 300 IU/ml) was perfused through the system over the next 24 hour period.

On Day 6, a sample was obtained for a cell count and fresh aMILs Base Medium supplemented with 0.02% Pluronic F68 and IL-2 (final concentration 300 IU/ml). Tubing was sterilely welded to the system so that over the subsequent 24 hours, 520 gms of media were perfused through the system. When necessary due to cell counts, Day 6 activities may be repeated for up to three additional days.

On the last day of expansion, the WAVE bag was removed from the system and samples obtained prior to manipulation for determination of viable cell content and mycoplasma contamination. The cell suspension was passed over the MagSep Magnetic Device to remove the ClinExVivo™ CD3/CD28 Paramagnetic Particles. Then, the cells were concentrated and washed using Plasma-Lyte A supplemented with 1% human serum albumin on the COBE 2991® Cell Processor. After washing, the product was sampled to determine the total number of viable cells. An aliquot of the cell suspension was microscopically examined for residual paramagnetic particle content. The cells were centrifuged to prepare for cryopreservation.

The aMILs Product was cryopreserved using a 6% hetastarch supplemented with 2% HSA and 5% DMSO. After the aMILs were suspended in the cryoprotectant, samples were removed for sterility and endotoxin testing. The aMILs Product was cryopreserved in AFC cryopreservation bags, along with QC vials and stored in vapor phase liquid nitrogen. At the time point defined in the clinical protocol, the bags were thawed in a 37±1° C. water bath at the patient's bedside and infused immediately.

### Example 20

### Specificity for Activated MILs

Previously it was shown that upon activation and expansion aMILs display a heightened specificity to myeloma plasma cells compared to un-activated MILs. A flow cytometric assay has been developed to evaluate this T-cell mediated tumor specificity of the aMIL products.

The test sample (aMILs product) was labeled with carboxyfluorescein diacetate succinimidyl ester (CFSE) according to manufacturer's protocol. CFSE consists of a fluorescent molecule containing a succinimydyl ester functional group and two acetate moieties. CFSE diffuses freely inside the cells and intracellular esterases cleave the acetate groups converting it to a fluorescent, membrane impermeable dye. This dye is not transferred to adjacent cells. CFSE is retained by the cell in the cytoplasm and does not adversely affect cellular function. During each round of cell division, relative fluorescence intensity of the dye is decreased by half and cellular division can be analyzed by the decrease of intensity of the cells in the FL-1 channel of a flow cytometer. Tumor specificity was determined by adding CFSE-labeled aMILs to irradiated HLA-matched allogeneic myeloma cell lines and examining the degree of CFSE dilution after a 72-hr co-culture.

### Example 21

### PDE-5 Inhibitor Drug Titration Protocol

The specific concentration of PDE-5 inhibitor to be used in the culture methods of the invention will vary depending on the specific PDE-5 inhibitor used. PDE-5 inhibitors can be tested for the optimal concentration of drug to be added to the cell culture system such that the drug is not toxic to the cells and is potent enough to inhibit MDSC suppression. Briefly, each PDE-5 inhibitor is added to culture media at several concentrations (e.g., fold dilutions, serial dilutions) and each of these concentrations is used in the expansion process as well as media alone as a control arm. The PDE-5 inhibitor is added on Day 0 of the expansion process.

Each day the cells are thoroughly mixed prior to removing the sample for analysis. The cells are monitored for both viability and CD3 expansion e.g., by Trypan-blue exclusion utilizing a hemacytometer and a microscope,viability=viable cells/viable cells+non-viable cells) and by CD3 antibody staining and analysis by flow cytometery. The total number of CD3⁺ cells is determined by multiplying the total number of cells by the percentage of CD3 cells as determined by flow cytometry.

The optimal PDE-5 inhibitor concentration is selected for promoting the greatest cell expansion with the least amount of cell death. This process should be repeated until the final day of expansion (typically day 5-8) and the concentration that shows the best potential (increased expansion, good viability) should be chosen. Note that this experiment should be repeated on several patient samples with varying amounts of MDSC's and that the MDSC's can be quantified by flow cytometry at the beginning of the process.

### Example 22

### Activated Marrow Infiltrating Lymphocytes in the Autologous Transplant Setting in Multiple Myeloma

To evaluate the clinical efficacy of activated marrow infiltrating lymphocytes (aMILs) administered alone or in combination with an allogeneic GM-CSF-based Myeloma Cellular Vaccine in patients undergoing an autologous hematopoietic stem/progenitor cell transplant for multiple myeloma.

Response Rates were evaluated utilizing the Blade' criteria, Complete Response (CR) rate, Near Complete Response (nCR) rate, Very Good Partial Response (VGPR) rate, Partial Response (PR) rate, Minimal Response (MR) rate, and Overall response rate (CR, VGPR, PR). Secondary Objectives include the evaluation of progression free survival and overall survival. Patients are monitored for progression/relapse on Days 60, 180, and 360, and as clinically indicated. Following one year follow-up, patients are followed every six months for the next four years.

To determine tumor-specific responses, tumor specific responses in blood and bone marrow are analyzed by examining T cell responses to DC-pulsed myeloma cell lines and induction of novel antibody responses. To evaluate the effect of aMILs on myeloma clonogenic precursors the side population of CD19 enriched PBLs throughout study are evaluated.

Patients interested in participating on this study sign consents. Eligibility is subsequently be determined based on the inclusion and exclusion criteria listed below. To be eligible, the patient must meet all of the following inclusion criteria previously determined for the trial including, but not limited to, previous diagnosis of multiple myeloma based on standard criteria, Durie-Salmon Stage II or III disease at any time since diagnosis; measurable serum and/or urine M-protein from prior to induction therapy documented and available; a positive serum free lite assay is acceptable; age at least 18 years old; ECOG performance status of 0-2; life expectancy at least 6 months; corrected serum calcium <11 mg/dL, and no evidence of symptomatic hypercalcemia. (Corrected serum calcium is calculated by adding 0.8 mg/dL to the measured serum calcium for every 1 g/dL that the serum albumin falls below 4.0 g/dL; serum total bilirubin and SGPT (ALT)<2.0 times the upper limit of normal; serum creatinine <2.0 mg/dL; and the ability to comprehend and have signed the informed consent

Exclusion criteria include diagnosis of any of the following cancers or any of the following conditions: POEMS syndrome (plasma cell dyscrasia with polyneuropathy, organomegaly, endocrinopathy, monoclonal protein [M-protein] and skin changes); non-secretory myeloma (no measurable protein on Serum Free Lite Assay); plasma cell leukemia; diagnosis of amyloidosis; previous hematopoietic stem cell transplantation; known history of HIV infection; in a complete remission at the time of bone marrow collection for MILs expansion; use of corticosteroids (glucocorticoids) within 21 days of vaccination; use of any myeloma-specific therapy within 21 days of vaccination; infection requiring treatment with antibiotics, antifungal, or antiviral agents within seven days of consent signing; participation in any clinical trial, within four weeks prior to vaccination or bone marrow collection on this trial, which involved an investigational drug or device; history of malignancy other than multiple myeloma within five years of consent signing, except adequately treated basal or squamous cell skin cancer; active autoimmune disease (e.g., rheumatoid arthritis, multiple sclerosis, systemic lupus erythematosis) requiring systemic treatment (hypothyroidism without evidence of Grave's Disease or Hashimoto's thyroiditis permitted).

Patients may be given a bone marrow transplant in conjunction with aMILs based therapy. Patients need to meet the criteria of The Johns Hopkins University to be eligible for transplant.

The aMILs product was administered intravenously (IV). The patient was pre-medicated with acetaminophen 650 mg by mouth and diphenhydramine hydrochloride 25-50 mg by mouth or IV, no greater than 30 minutes prior to the infusion of aMILs. These medications were repeated every six hours as needed. A course of non-steroidal anti-inflammatory medication may be prescribed if the patient continues to have a high fever not relieved by acetaminophen.

Other medications may also be prescribed to treat additional side effects during the course of treatment. However, there are some medications and therapies that may have adverse effects on the activity of aMILs therapy and therefore are contraindicated. In particular, patients should not receive systemic corticosteroids such as hydrocortisone, prednisone, prednisolone (Solu-Medrol) or dexamethasone (Decadron) at any time, except in the case of a life-threatening emergency, since this may have an adverse effect on aMILs. If steroids are required for an acute infusion reaction, an initial dose of hydrocortisone 100 mg is recommended.

The patient is hydrated with D5^{1/2}NS at approximately 200 mL per hour for at least one hour prior to infusion. If the aMILs bag is damaged, or otherwise compromised, it is not be infused. Otherwise the aMILs are thawed and infused through standard blood tubing without an additional filter into a peripheral or central IV site. Each of the bags is infused at a rate of approximately 10 mL per minute. Following aMILs infusion, the patient is hydrated with D5W1/2NS at approximately 200 mL per hour for two hours.

The dose of aMILs administered will vary based on the number of cells expanded. All aMILs will be infused except those used for QC testing.

The aMILs product is administered once.

Activated MILs are labeled with CFSE (Invitrogen, Carlsbad, Calif. cat. No. C34554) and suspended at a cell concentration of 1×10⁶ cells/ml to confirm tumor specificity prior to use. The CFSE-labeled aMILs are cultured under three conditions in a 96-well plate:

a. aMILs (1×10⁵ cells) with 100 ul of media (background control)
b. aMILs (1×10⁵ cells) with 100 ul containing 1×10⁵ irradiated H929 cells (myeloma tumor cell line)
c. aMILs (1×10⁵ cells) with 100 ul containing 1×10⁵ irradiated U266 cells (myeloma tumor cell line).

The plates were incubated at 37° C. for up to 72 hours. Cells were harvested and labeled with CD3-APC according to the manufacturer's protocol (BD Biosciences San Diego, Calif. cat. No. 555342) and analyzed using a flow cytometer.

CD3⁺ cells were gated on and CFSE dilutions analyzed based on the cells in this gate. A percentage ≧1.5 fold above background for the aMILs incubated with H929 and U266 cells are considered a tumor-specific or positive result.

### Sample Size

A total of 20 evaluable patients were enrolled in this clinical trial. If patients were registered but did not receive the aMILs cells for any reason, then additional patients were registered to ensure 20 patients were treated. The choice of sample size 20 was mainly based on the nature of the feasibility study.

The primary outcomes of this study are feasibility, clinical efficacy and toxicity. Feasibility is assessed qualitatively based on patient accrual, ability of patients to adhere to study schema, drop-out, and protocol violations. The clinical efficacy is defined as complete response based on Blade's Criteria.

The primary safety endpoints were treatment-related mortality and the incidence of Grade 3 hematologic toxicity. Toxicity is graded using the NCI Common Toxicity Criteria (Version 3.0; http://ctep.cancer.gov/reporting/ctc.html). Treatment related mortality was defined as death not attributable to disease progression/relapse (and excluding the ‘unrelated’and ‘not applicable’ categories in attribution of causality as determined using standard clinical measures) that occurs within the first 100 days following transplantation.

Toxicity was monitored closely and no aMILs related toxicity was observed.

Disease progression was defined using standard clinical measures. Progression-free survival was defined as time from date of randomization to the date of first observation of disease progression or death due to disease progression. Overall survival was defined as the time from the date of randomization to date of death due to all causes. The Probability of progression-free survival (PFS) and overall survival (OS) is estimated using the Kaplan-Meier method and displayed graphically. Median PFS and OS will be reported along with 95% confidence intervals. Proportion of patients who were PFS and OS at specific time of follow-up will be reported as well.

Statistical analyses of other secondary objectives such as T cell reconstitution, anti-tumor immune response, tumor-specific vaccine response, effect of aMILs on osteclastogenesis, and Myeloma clonogenic precursors are considered exploratory. Descriptive statistics will be provided. All data will be summarized based on nature distribution of data and presented separately by treatment group at clinical defined time points, or by primary clinical response.

The association between clinical responses and in vitro measurements of T cell laboratory correlates, including T cell proliferation to tumor and ELISPOT analysis of both peripheral as well as marrow infiltrating lymphocytes will be explored by using multivariate regression models as appropriate.

### Hematopoietic Engraftment:

The mean and median time to neutrophil and platelet engraftment (absolute neutrophil count >500/mm³ and platelet >20,000/mm³) will be summarized among those with successful engraftment, along with the rate of primary graft failure. It should be remembered that in the absence of post-transplant G-CSF, we would expect neutrophil engraftment to be slightly delayed. However, should red cell, platelet, and neutrophil engraftment all show evidence of significant delay in more than 2 patients, the study will be placed on hold until a more formal evaluation can be performed.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method for expanding marrow infiltrating lymphocytes (MILs) comprising:
obtaining bone marrow from a subject comprising a malignant cancer;
contacting the bone marrow with a PDE-5 inhibitor; and
contacting the bone marrow with anti-CD3 and anti-CD-28 antibodies, wherein the aMILs are expanded at least 30-, 40-, 50-, 60-, 70-, 80-, 90-, 100-fold, or more.

2. The method of claim 1, wherein the cancer comprises a hematological malignancy.

3. The method of claim 1, wherein the bone marrow is contacted with a single dose of a phosphodiesterase (PDE)-5 inhibitor.

4. The method of claim 1, wherein the bone marrow is contacted with the anti-CD3 and anti-CD-28 antibodies for 5-14 days.

5. The method of claim 1, wherein after contacting the bone marrow with the anti-CD3 and anti-CD28 antibodies, the antibodies are removed.

6. The method of claim 1, wherein the malignant cells are not expanded.

7. The method of claim 1, wherein the number of malignant cells in the population is decreased by at least 50%, 60%, 70%, 80%, 90%, or 95% as compared to the number of malignant cells present in the bone marrow population prior to culturing as determined by flow cytometry staining with an anti-CD30 antibody.

8. The method of claim 1, wherein the malignant cells are below the level of detection as determined by flow cytometry.

9. The method of claim 1, wherein expansion of MILs is at least 30%, 40%, 50%, 60%, 70%, 80%, 90%, or 100% greater in a culture contacted with a PDE-5 inhibitor as compared to a culture not contacted with a PDE-5 inhibitor.

10. The method of claim 1, further comprising providing a rigid, stable, round bottom, closed container of sufficient volume for expanding bone marrow in a volume of at least 20 ml of cells and growth media.

11. The method of claim 1, wherein the bone marrow is treated to substantially remove the neutrophils and red blood cells from the bone marrow prior to contacting the bone marrow with the anti-CD3 and anti-CD-28 antibodies.

12. The method of claim 11, wherein the neutrophils and red blood cells are substantially removed by density centrifugation

13-18. (canceled)

19. A method for the treatment of cancer comprising administering aMILs of claim 1 to the subject from which the bone marrow was obtained.

20. The method of claim 19, wherein the cancer comprises a hematological malignancy.

21. The method of claim 19, wherein the MILs are administered once.

22. The method of claim 1, further comprising administration of a bone marrow transplant.

23. The method of claim 1, further comprising lymphoablation prior to administration of MILs.

24. (canceled)

25. A composition prepared by the method of claim 1 in a pharmaceutically acceptable carrier.

26. A device for culturing cells for administration to a human subject comprising:
an enclosed cell culture container comprising a smooth, rigid, rounded bottom surface;
a first port and a second port operably linked to the cell culture container; and
a support to maintain the cell culture container in a fixed position to allow for stationary cell culture.

27-34. (canceled)

35. A method of culturing cells comprising:
providing a device of claim 26;
introducing cells into the cell container;
introducing media into the cell container,
introducing air into the cell container to make the cell container rigid;
placing the cell container on the support; and
culturing the cells in a static culture.
